(19) United States
(12) Patent Application Publication
(10) Pub. No.: US 2021/0035679 A1
(43) Pub. Date: Feb. 4, 2021

(54) SOCIAL DETERMINANTS OF HEALTH SOLUTION

(71) Applicant: Experian Health, Inc., Franklin, TN (US)

(72) Inventors: Mindy Pankoke, Lincoln, NE (US); Karly Marie Rowe, Scottsdale, AZ (US); Christopher Busch, Maple Grove, MN (US); Andrew Jonathan Levitte, Scottsdale, AZ (US); Rachel Dana Goudie, Franklin, TN (US); Dimuthu Wijetilleke, Atlanta, GA (US); Matthew McCawley, Marietta, GA (US); Dusty Cheyenne Patterson, Smyrna, TN (US)

(73) Assignee: Experian Health, Inc., Franklin, TN (US)

(21) Appl. No.: 16/916,747

(22) Filed: Jun. 30, 2020

Related U.S. Application Data
(60) Provisional application No. 62/880,408, filed on Jul. 30, 2019.

(51) Int. Cl. G16H 40/20; G06Q 30/02; G16H 50/30; G16H 50/20; G16H 10/60; G16H 50/70; G06F 16/9535

(52) U.S. Cl. CPC G16H 40/20; G06Q 30/0201; G16H 50/30; G06Q 10/105; G16H 10/60; G16H 50/70; G06F 16/9535; G16H 50/20

(57) ABSTRACT

Identifying and addressing an individual's socio-economic factors related to negative health outcomes for reducing the likelihood of negative health outcomes for the individual in the form of readmission rates, emergency department utilization, missed appointments/no-shows, etc., may be realized by employing aspects of the present disclosure. A social determinants of health (SDoH) system accesses and analyzes consumer marketing data associated with the individual, business data, and public transportation data held by various databases for identifying indicators of SDoH factors, determines an individual's propensity for SDoH factors, and determines an engagement strategy to address SDoH that impact the individual. The results may be included in a report or included in a user interface that is transmitted to the requestor. The results may be displayed in a way that enables the requestor to easily identify and act on the individual's SDoH using recommended engagement strategies.

### CROSS-REFERENCE TO RELATED APPLICATIONS

This application claims the benefit of U.S. Provisional Application No. 62/880,408, having the title of “Social Determinants of Health” and the filing date of Jul. 30, 2019, which is incorporated herein by reference in its entirety.

### BACKGROUND

Health outcomes of a population are affected by a variety of medical and non-medical factors. Example non-medical factors may include socioeconomic factors and environmental factors, and may account for a large proportion of modifiable contributors to health outcomes for a population. For example, excessive healthcare utilization, missed appointments, or other negative outcomes may be due in part to medical conditions; however, non-medical socioeconomic factors, such as whether a person has means to transportation to a healthcare provider, means to healthful food options, stable housing, etc., may additionally or alternatively be linked to negative or high health outcomes. Such socioeconomic situations as these and others can result in higher healthcare costs, not only for patients, but also for healthcare organizations.

Non-medical factors that may affect health outcomes may be referred to as social determinants of health (SDoH). Current efforts by healthcare organizations in attempting to solve problems encompassing SDoH as they relate to negative/high health outcomes are elementary in their approach, are manual and time-consuming, do not produce holistic, accurate, or actionable data, and/or are expensive to implement.

For example, one current approach used by healthcare organizations is to survey patients. Patient surveying is oftentimes performed by asking patients questions that aim to identify an SDoH. Results of patient surveys are contingent on the patients' responses, which can be inaccurate due to a patient's perspective or truthfulness, or due to a lack of completeness, where patients may opt out of answering certain questions. Additionally, surveys are constrained to a finite number of questions, which may not be robust enough to be impactful, and surveying patients is associated with high costs in physicians' time and other resources for data input and analysis.

Some current approaches to solve problems encompassing SDoH as they relate to negative/high health outcomes may produce a score tied to a specific area (e.g., readmission risk, total cost, access to transportation, housing instability). While a score may give some context to a patient's propensity to incur negative health impacts due to SDoH, this score may be based on inaccurate or constrained data. Such a score may be an ill-representation of patients without further detail and may not be sufficient for healthcare professionals to trust. Moreover, a score is not actionable. That is, a score may not provide a healthcare organization with information that can help them to make informed decisions or for informing the organization about how to appropriately engage patients.

Another example current approach used by healthcare organizations is the use of clinical/medical data to infer SDoH. However, inferring SDoH from clinical/medical data may only help to identify medical-related contributors to health outcomes while omitting non-medical contributors. As stated above, non-medical factors, which are not accounted for in clinical/medical data, may contribute to a large proportion of health outcomes. As can be appreciated, this approach lacks visibility of non-medical factors, thus presenting a fragmented view of a patient's SDoH that may produce insufficient results for identifying and/or addressing SDoH that contribute to negative/high health outcomes.

Another way in which healthcare organizations are trying to solve problems encompassing SDoH as they relate to negative/high health outcomes is by taking a blanket approach to partner with or invest in programs that aim to address SDoH. For example, healthcare organizations may offer enrollment into social-community programs (e.g., client-specific programs, vendor-provided programs, public programs) that may address patient propensities, or may contribute to establishing transportation, counselling, and/or grocery stores in obvious impoverished areas. As can be appreciated, blanket solutions are not only expensive, but may lack socioeconomic insights that should drive an approach for a patient. For example, without the knowledge of “who” should be using these programs, those with SDoH that relate to negative/high health outcomes may not use the programs and may continue to have excessive healthcare utilization and costs.

### SUMMARY

Aspects of methods, systems, and computer-readable storage devices for providing a Social Determinants of Health (SDoH) solution are provided herein. The SDoH propensity solution described herein provides deep insight into non-clinical factors that impact healthcare outcomes and provides actionable information to healthcare organizations that can reduce negative/high health outcomes such as readmission rates, emergency department utilization, missed appointments/no-shows, etc.

According to an aspect, the SDoH system is configured to receive, from a requestor, demographic information corresponding to an individual. For example, the demographic information may be related to the individual and provided as part of a request for SDoH information. Responsive to receiving the demographic information, the SDoH system is configured to use the demographic information to determine a unique identifier corresponding to the individual, and then use the unique identifier to obtain relevant socioeconomic data related to the individual from a multi-source database. The SDoH system is further configured to obtain business data related to health-related businesses near the individual from a business database and public transportation data from a public transportation database, among other databases housing SDOH-relevant data such as food bank locations, homeless shelter locations, and more.

The obtained data, which may include other data obtained from other databases, may be used to identify various indicators of SDoH factors that may contribute to the individual's propensity for negative health outcomes. A scoring engine may be used to generate a propensity score and level for the individual that indicates the individual's propensity to incur negative health impacts due to one or more SDoH factors. For example, the propensity score may be computed based on various SDoH factor indicators that are identified in an analysis of the obtained data associated with the individual. Further, a justification engine may be used to generate a human-readable justification and justification details that provide insight into the indicators and factors that drive the propensity score. Additionally, a rules engine may be used to determine a relevant and appropriate engagement strategy for addressing the individual's SDoH. According to an aspect, an SDoH propensity solution, which may include the propensity score, propensity level, justification, justification details, and engagement strategy, may be generated for the individual and included in a report and/or included in a graphical user interface that may be generated and transmitted to the requestor for display on a computing device. In some implementations, the SDoH propensity solution may include visualizations of aggregate levels of SDoH propensities for various segments of individuals or the population. According to an aspect, the SDoH propensity solution provides a comprehensive and easily-understandable view of SDoH that impact an individual and the indicators of the SDoH. Moreover, the engagement strategies that may be included in the SDoH propensity solution provide the requestor with one or more effective types of engagement with an individual to positively impact the individual and reduce the likelihood of negative health outcomes, such as in the form of readmissions, no-shows, emergency department usage, etc.

### BRIEF DESCRIPTION OF THE DRAWINGS

The accompanying drawings, which are incorporated in and constitute a part of this disclosure, illustrate various aspects and examples of the present invention. In the drawings:

FIG. 1 is a block diagram illustrating an example operating environment for implementing aspects of the present disclosure;

FIG. 2 is a block diagram illustrating components of a social determinants of health (SDoH) system with which aspects of the present disclosure may be practiced;

FIG. 3 is a flow diagram illustrating an example process of mapping an individual defined in an SDoH request to consumer marketing data according to an embodiment;

FIG. 4 is a flow diagram illustrating an example process of determining an SDoH propensity score and level, a justification for the propensity score and level, justification details associated with the justification, and an engagement strategy for addressing SDoH factors indicated by the score, level, justification, and/or justification details;

FIG. 5 is an illustration of example SDoH propensity solution results provided in a batch report;

FIGS. 6A-F are illustrations of example user interfaces that may be generated for displaying SDoH propensity solution results;

FIG. 7 is a flow chart showing general stages involved in an example method for generating and providing SDoH propensity solution results; and

FIG. 8 is a block diagram illustrating physical components of an example computing device with which aspects may be practiced.

### DETAILED DESCRIPTION

The following detailed description refers to the accompanying drawings. Wherever possible, the same reference numbers are used in the drawings and the following description to refer to the same or similar elements. While aspects of the present disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the present disclosure, but instead, the proper scope of the present disclosure is defined by the appended claims. Examples may take the form of a hardware implementation, or an entirely software implementation, or an implementation combining software and hardware aspects. The following detailed description is, therefore, not to be taken in a limiting sense.

The present disclosure provides a system, method, and computer readable storage device including computer readable instructions, which when executed by a processing unit, provide a Social Determinants of Health (SDoH) solution. Aspects of the SDoH propensity solution described herein provide deep insight into non-clinical factors that impact healthcare outcomes and provide actionable information to healthcare organizations that can reduce negative/high health outcomes such as readmission rates, emergency department utilization, missed appointments/no-shows, etc. Although examples are given herein primarily involving healthcare providers and patients, it will be recognized that the present disclosure is applicable to other types of service providers who provide services to clients, the outcome of which may be impacted by the client's SDoH. As such, the terms “user,” “patient,” and “client” may be used interchangeable herein.

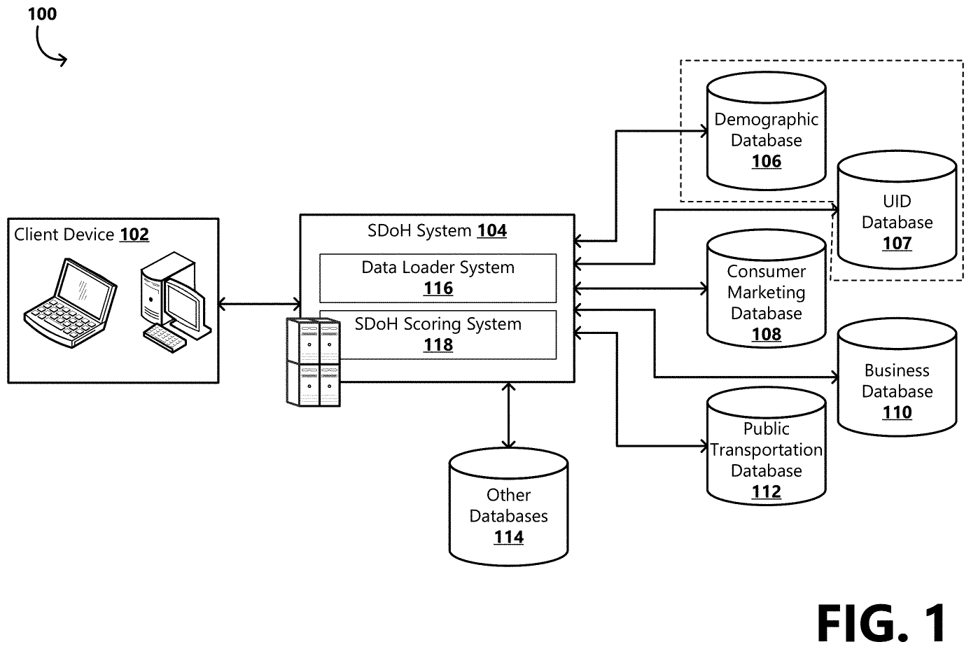

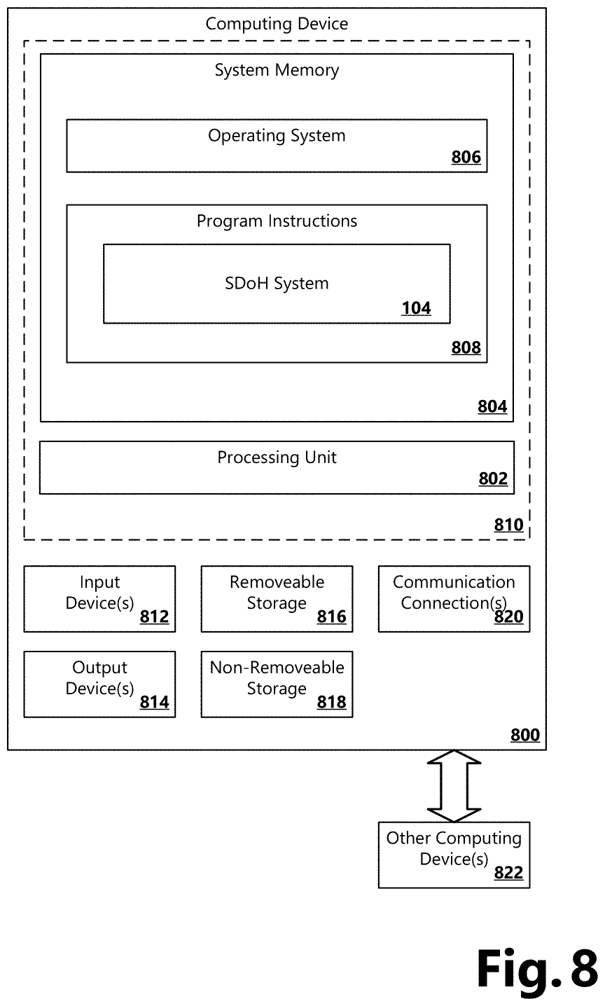

FIG. 1 is a block diagram illustrating an example operating environment 100 in which various aspects of an SDoH system 104 may operate. As illustrated, the SDoH system 104 is in communication with one or more client devices 102, a demographic database 106, a unique identifier (UID) database 107, one or more consumer marketing databases 108, one or more business databases 110, one or more public transportation databases 112, and in some implementations, one or more other databases 114. In various implementations, SDoH system 104 is implemented as part of or communicatively attached to an Identity Management System that is configured to accurately identify individuals and match records within and across disparate healthcare organizations (e.g., pharmacy, lab, payer, and provider), and facilitate information exchange within and across the healthcare ecosystem using a unique, universal person/patient identifier (UPI). Each of the SDoH system 104, the one or more client devices 102, the demographic database 106, the consumer marketing databases 108, the one or more public transportation databases 112, and one or more other databases 114 may be implemented on or by one or more computing devices 800 discussed in greater detail in regard to FIG. 8. The computing devices include, but are not limited to: servers, desktop computers, laptops computers, tablets, smart phones, personal digital assistants, and distributed systems that are run on multiple computing devices. Although not illustrated, one of ordinary skill in the art will appreciate that various intermediary computing and networking devices may exist between the illustrated elements of the operating environment 100 to facilitate communications between the various enumerated elements, for example via the Internet and one or more Intranets.

The SDoH system 104 provides clients a turnkey SDoH propensity solution that, in response to receiving a request from a client device 102 for SDoH information for an individual, utilizes a data loader system 116 to obtain socioeconomic/environmental data, and/or personal/behavioral factors relating to the individual and/or the individual's household, and uses an SDoH scoring system 118 to determine a propensity score and level for the individual that indicates the individual's propensity to incur negative health impacts due to SDoH, a justification and justification details for the score/level, and determine a patient engagement strategy based on the justification and justification details, and provides an SDoH propensity solution in a response to the client device that includes the propensity score, justification, justification details, and the patient engagement strategy. The individual's SDoH propensity solution may be delivered in a format (as will be described in further detail below) that clearly indicates the individual's propensity for negative/high SDoH-related health outcomes, includes insights into the factors that drive the individual's determined propensity, and patient engagement strategies that provide the client with actionable information to reduce the likelihood for negative health outcomes, such as readmissions, no-shows, emergency department usage, and the like.

The client device 102 is illustrative of a computing device of a requesting client seeking SDoH data related to an individual. In various aspects, the client device 102 runs a specific program (e.g. client application) to access the SDoH system 104. In other aspects, the client device 102 accesses the SDoH system 104 via an Application Program Interface (API) 216. For example, the SDoH system 104 may expose one or more APIs 216 that may integrate with one or more software platforms, applications, and/or health information systems executing on the client device 102. In other aspects, the client device 102 accesses the SDoH system 104 via a thin client that is configured to request and return the SDoH data via a web browser. According to an example, the client device 102 is associated with a healthcare organization that provides healthcare services to individuals.

The demographic database 106 is illustrative of a computing device configured to store demographic data used for patient matching. Demographic data may include, but are not limited to: name, date of birth, address, identifier numbers, known associates (e.g., employer, spouse, parent), telephone number, email address, etc. In various aspects, the demographic database 106 is a relational database. Demographic data stored by the demographic database 106 may be used by the SDoH system 104 to identify an individual uniquely based on demographic data in an SDoH information request received from a client device 102 matching demographic data held by the demographic database 106.

The UID database 107 is illustrative of a computing device configured to store UIDs that are associated with various entities. For example, the UIDs stored in the UID database 107 may comprise a plurality of UIDs used by various data sources and/or clients to uniquely distinguish individuals and to store and access data records in association with those individuals the data records describe. In some examples, the UID database 107 stores UIDs from data records that have been mapped based on a determination that the individuals described in those data records match. In some implementations, the UID database 107 is a relational database where a person has multiple UIDs associated with him/her, but a particular UID is only associated with one person. According to an aspect, the UID database 107 may be used to look up a consumer data UID (CD-UID) for an individual corresponding to a known UID (universal person/patient identifier (UPI)) for the individual. For example, the UPI may be a universal identifier that is used to link various data records from a plurality of data sources that correspond to an individual. The UPI may be used internally by the SDoH system 104 and affiliated systems (e.g., the Identity Management System within which the SDoH system may be implemented or with which the SDOH system may be communicatively attached), and may further be shared with and used by contributors of data stored and/or managed by the SDoH system 104 and affiliated systems. In some examples, the demographic database 106 and the UID database 107 may be combined as a single database (e.g., the demographic database 106 may include UIDs for individuals in addition to demographic data of those individuals.

The consumer marketing database 108 operates as a source of consumer data related to an individual and/or the individual's household to the SDoH system 104. For example, the data loader system 116 of the SDoH system 104 may query the consumer marketing database 108 for certain consumer related data that are used by the SDoH scoring system 118 of the SDoH system (in addition to other as will be described below) to determine an individual's propensity to incur negative health impacts due to SDoH. The data loader system 116 of the SDoH system 104 is therefore configured or operable to query the consumer marketing database 108 for records that are stored by the consumer marketing database based on an identifier of an individual matching demographic data held by the demographic database 106.

The consumer marketing database 108 is illustrative of a computing device configured to store a compilation of various public and proprietary consumer data, including data related to consumer socio-demographic, lifestyle, culture and behavior. For example, the consumer marketing database 108 may comprise data records such as, but not limited to: demographic data (e.g., age, gender, marital status, children and income), customer/consumer data (e.g., purchase data, consumer decision-making styles), marketing data (e.g., preferred marketing communication channels), name and address information, observed and self-reported lifestyle and other behavioral data interests, hobbies and brand preferences), public record information, realty and property tax information, automotive data, financial data, credit information, census data, and so on. One example of such an inclusive database, which may comprise much of the above-mentioned types of data for approximately 98% of U.S. individuals and living units (households), is the EXPERIAN CONSUMERVIEW database. For example, the consumer marketing database 108 may comprise thousands of data points on each of millions of consumers and households.

In various aspects, the consumer data are provided by direct data sources, that is, data sources who have direct relationships with consumers who opt in to their data collection. As part of collecting data from direct data sources, the consumer marketing database 108 is configured to be continually updated with new/fresh data from a plurality of data sources. In some examples, new/fresh data may be received from various direct data sources twenty-hours per day and seven days each week. The consumer marketing database 108 may include a database management engine that evaluates business rules that ensure data elements that are housed within the consumer marketing database 108 are accurate. Further, the consumer marketing database 108 is configured to attach a unique identifier (herein referred to as a consumer identifier (CID) to each consumer record, which provides for a stable and consistent repository of consumer data. In various aspects, the consumer marketing database 108 provides improved data security by storing source codes on each stored data record, wherein the source code points to the original data sources and their respective consumer privacy policy. Accordingly the consumer marketing database 108 is configured to supply data to the SDoH system 104 in compliance with the original data sources' policies, thus protecting consumers' privacy.

The business database 110 operates as a source of data of healthcare related businesses. For example, the business database 110 is illustrative of a computing device configured to store data on businesses related to healthcare, such as clinics, hospitals, healthcare provider offices, pharmacies, rehabilitation clinics, grocery stores, etc. The data loader system 116 of the SDoH system 104 may query the business database 110 for information associated with healthcare related businesses, such as types of healthcare related businesses and their locations. This information may be used for determining an individual's proximity to such locations as part of determining the individual's propensity to incur negative health impacts due to SDoH.

The public transportation database 112 operates as a source of public transportation data that the data loader system 116 of the SDoH system 104 is configured to access. For example, the public transportation database 112 is illustrative of a computing device configured to store data on transit systems stops and routes. The public transportation stored by the public transportation database 112 may be obtained from various original sources, such as national, state, and/or regional transit map databases, individual transit operator sources, etc. The public transportation database 112 may store data on locations of bus stops, train stations, subway stations, and the like. The SDoH system 104 may query the public transportation database 112 for locations of public transportation sites as part of determining an individual's propensity to incur negative health impacts due to SDoH. According to an aspect, the data loader system 116 of the SDoH system 104 may be configured or operable to query other databases 114 for additional data that can be used by the SDoH system to identify possible SDoH that may negatively affect individuals.

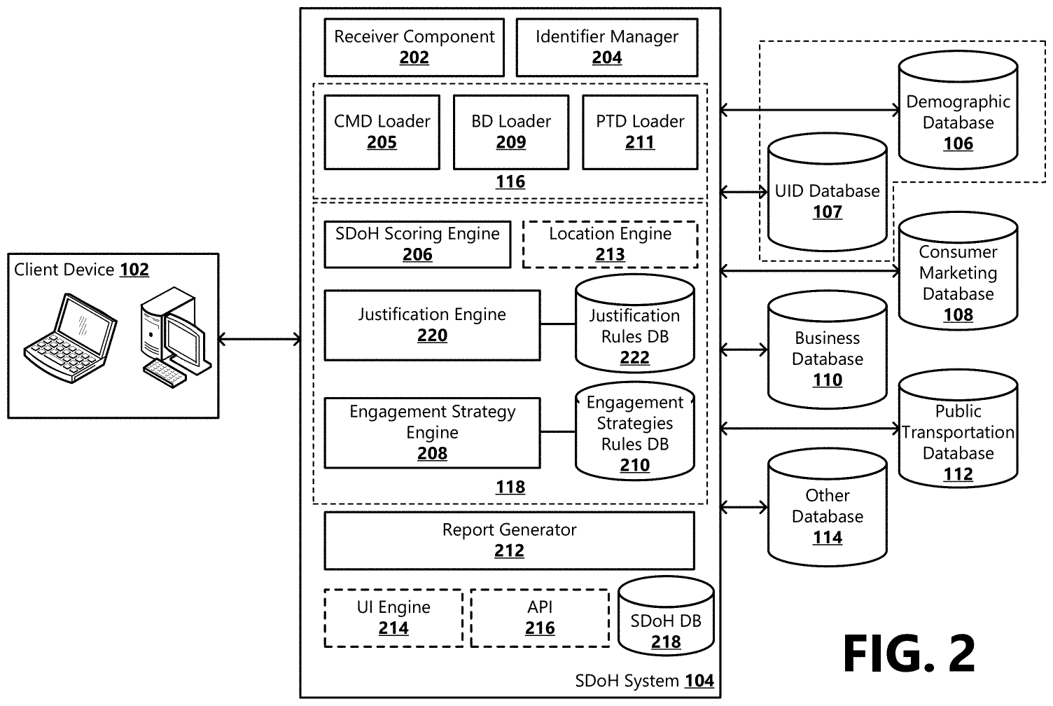

With reference now to FIG. 2, a block diagram of an example SDoH system 104 is illustrated. To determine and provide client devices 102 SDoH propensity solutions, the SDoH system 104 includes various subsystems or components, including: a receiver component 202, an identifier manager 204, the data loader system 116 including a consumer marketing data loader 205, a business data loader 209, and a public transportation data loader 211, the SDoH scoring system 118 including an SDoH scoring engine 206, a justification engine 220, a justification rules database 222, an engagement strategies engine 208, and an engagement strategies database 210, a location engine 213, a report generator 212, an optional user interface (UI) engine 214, optional APIs 216, and an SDoH database 218. In some examples, one or more components or subsystems may be combined. In other examples, each of the components or subsystems may be implemented as one or more separate computing devices, software applications, program modules, or the like.

In some aspects, the SDoH system 104 maintains the demographic database 106 as a subsystem, while in other aspects, the SDoH system 104 is in communication with an externally managed demographic database 106. In some aspects, SDoH system 104 maintains the consumer marketing database 108 as a subsystem, while in other aspects, the SDoH system 104 is in communication with an externally managed consumer marketing database 108. In some aspects, SDoH system 104 maintains the business database 110 as a subsystem, while in other aspects, the SDoH system 104 is in communication with an externally managed business database 110. In some aspects, SDoH system 104 maintains the public transportation database 112 as a subsystem, while in other aspects, the SDoH system 104 is in communication with an externally managed public transportation database 112. Each of the databases may be implemented as one or more separate computing devices, software applications, program modules, or the like.

According to an aspect, the various processes that implement aspects of the present disclosure may be initiated when a client device 102 sends an SDoH request to the SDoH system 104. The client device 102 may be implemented within a healthcare organization system; and by sending the SDoH request to the SDoH system 104, the client (e.g., such as a hospital, clinic, doctor's office) may wish to gain insight into non-clinical factors that may impact the healthcare outcome of an individual (e.g., a patient of the client) and patient engagement strategies that may address the individual's SDoH. The client device 102 may include an application, a device running an application, a legacy device attached to a system with an application, and the like. The client device 102 may initiate the SDoH request, which is transmitted to the SDoH system 104 via a secure transfer protocol, and received by the receiver component 202 of the SDoH system 104. The SDoH request may be sent to the SDoH system 104 in various ways. In some aspects, the SDoH request is sent in one or more batch transactions. In other aspects, the SDoH request is transmitted by a user agent, which can include a specific program (e.g. client application) configured to access the SDoH system 104 or a thin client configured to request and return SDoH information via a web browser. In other aspects, the SDoH system 104 may expose one or more APIs 216 that may integrate with one or more software platforms, applications, and/or health information systems operating on the client device 102. For example, the one or more APIs 216 may be used to transmit an SDoH request to the SDoH system 104 for requesting SDoH propensity information for an individual on demand.

The receiver component 202 is configured or operable to receive an SDoH request and send the request to the identifier manager 204. According to various aspects, an SDoH request includes demographic data corresponding to one or more individuals for whom SDoH propensity information is requested. The demographic data may be used by the identifier manager 204 to correlate an individual to a unique identifier (UID) that can be used to obtain, from the consumer marketing database 108, consumer marketing data corresponding to the individual associated with the UID. In some examples, the receiver component 202 is configured or operable to extract specific demographic data from an SDoH request, and pass the extracted demographic data to the identifier manager 204. In other examples, the receiver component 202 may be further configured or operable to convert demographic data that are received in the SDoH request from the format in which they are received to a standardized format used by the identifier manager 204. For example, demographic data fields in the received SDoH request may be rearranged, merged, split, or omitted to normalize them, truncated (e.g., nine digit ZIP codes to five digit ZIP codes), etc.

The identifier manager 204 is configured or operable to identify an individual based on identifying data 306 (e.g., demographic data) included in the SDoH request, and to create a three-way mapping between the identified individual to a UPI and further to a CD-UID for obtaining data used by the SDoH system 104 to determine the individual's SDoH propensity. According to an aspect, the UPI may be an identifier assigned to an individual by the entity performing the mapping. In some examples, the UPI may be used to link data records associated with the same individual within and across disparate healthcare organization systems. The CD-UID is an identifier assigned to the same individual and used by the consumer marketing database 108 to store consumer marketing data and indicators associated with the individual. The three-way mapping enables linking the SDoH request and the associated individual to demographic data stored in the demographic database 106 (and optionally to healthcare related data for the individual collected from one or more disparate healthcare organization systems) and to consumer marketing data stored in the consumer marketing database 108.

In various aspects, the identifier manager 204 works in conjunction with the demographic database 106 to identify a given individual uniquely based on client-supplied demographic data in the SDoH request matching demographic data stored by the demographic database 106. Demographic data may include, but are not limited to: name, address, date of birth, other identifier numbers, known associates (e.g., employer, spouse, parent), telephone number, email address, etc. In various aspects, the identifier manager 204 uses a probabilistic matching algorithm to determine whether the information included in an SDoH request indicate whether the person described therein is a person who has previously been associated with a UPI (and if so, what the UPI is). The UPI may then be mapped to the SDoH request and to the associated individual identified in the request. In some examples, the identifier manager 204 may be further operable or configured to make a determination to create a new UPI if the described person in the SDoH request is not associated with prior-gathered data records. In some examples, the demographic data used by the identifier manager 204 may be shared with the demographic database 106 so that as demographic details for a given person are observed (e.g., a change of address or name, a misspelling/mis-entry of a data field), the demographic database 106 may store those data for later matching to identify the person again in the future.

In various aspects, the identifier manager 204 works in conjunction with the UID database 107 to match the UPI associated with the individual described in the SDoH request with a CD-UID stored in the UID database 107. As described above, the UID database 107 may store a plurality of UIDs for the individual, which may include a universal UID and/or a plurality of entity-specific UIDs (e.g., specific to the client from which the SDoH request is received, specific to other clients, specific to the SDoH system 104, specific to the consumer marketing database 108, specific to other databases 114). The identifier manager 204 may query the UID database 107 for or may perform a lookup operation to look up the CD-UID for the known UPI associated with the individual. The CD-UID associated with the UPI in the UID database 107 may then be mapped to the SDoH request and to the associated individual identified in the request. As described above, the CD-UID is an identifier used by the consumer marketing database 108 to uniquely identify individuals for whose consumer data and indicators are stored by the consumer marketing database. Accordingly, CD-UID mapped to the individual associated with the SDoH request can be used by the SDoH system 104 to obtain consumer marketing data stored in the consumer marketing database 108 for the individual for determining the individual's SDoH propensity. The UPI and CD-UID for the individual may be passed to the SDoH scoring engine 206.

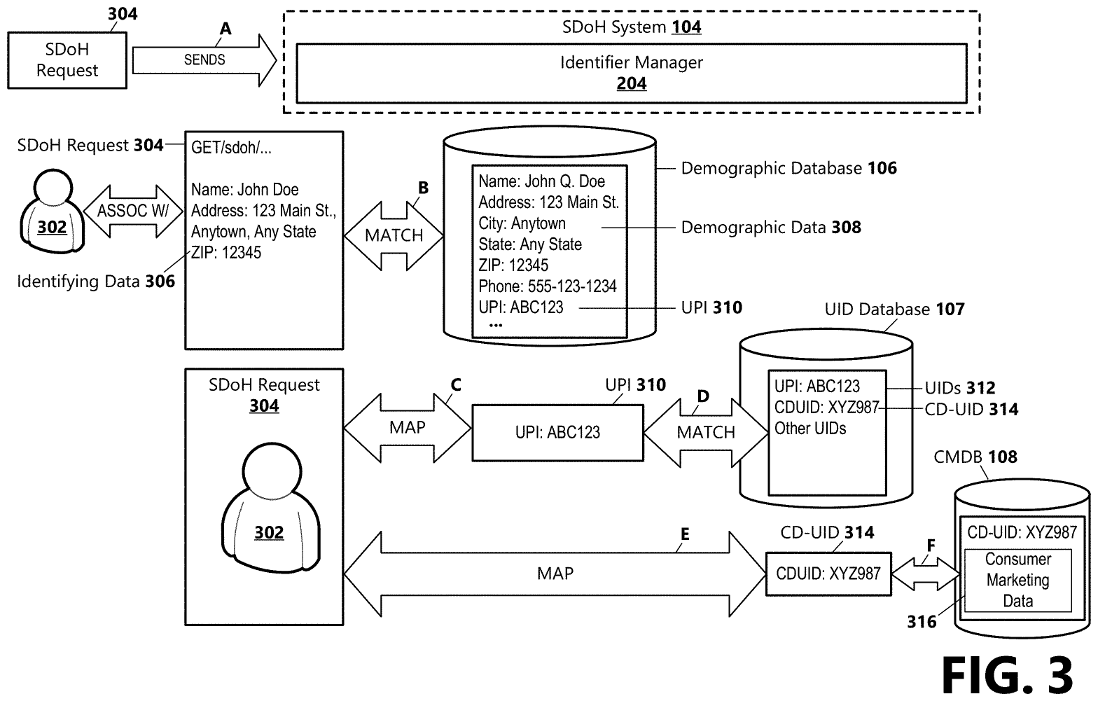

FIG. 3 is a flow diagram illustrating an example process of mapping an individual 302 defined in an SDoH request 304 to consumer marketing data 316 according to an embodiment. With reference now to FIG. 3, the process is initiated when a client device 102 sends an SDoH request 304 to the SDoH system 104, as shown by the letter A. The SDoH request 304 includes client-supplied identifying data 306 that identify an individual 302. The identifying data 306 are to be matched, by the identifier manager 204, with demographic data 308 that are stored in the demographic database 106. The identifying data 306 may include data that define/describe the individual 302, such as demographic data that may be entered into the client system, such as when an individual (e.g., patient) visits the client (e.g., healthcare provider) to receive healthcare services and provides his/her demographic information to an administrative worker or input interface. As shown, the example identifying data 306 include the individual's name, an address for that individual 302, and the individual's date of birth. These data fields are provided herein for illustrative purposes only; additional and/or alternative data fields may be used in various aspects for matching.

As described above, the demographic database 106 stores demographic data 308 about individuals. For example, the demographic database 106 may store demographic data 308 corresponding to the individual 302 defined/described by the identifying data 306 included in the SDoH request 304. The identifier manager 204 is configured to match the individual's information as indicated by the identifying data 306 to the individual's information as indicated by the demographic data 308 stored in the demographic database 106, as shown by the letter B. In an example, the match may be performed on the basis of the individual's name, address, and date of birth information. However, in various embodiments, the matching may be based on other pieces of information. On the basis of the match, a mapping between the UPI 310 associated with the individual's demographic data 308 and the SDoH request 304 and the individual 302 described in the request is created, as shown by the letter C.

The identifier manager 204 is further configured to perform a UID match, as shown by letter D. For example and as shown in FIG. 3, the UPI 310 for John Doe (“ABC123”) is matched with the CD-UID 314 (“XYZ987”) stored in the UID database 107. With the identifying data 306 to UPI 310 mapping having been created, a three-way ID mapping enables the SDoH system 104 to link consumer marketing data 316 assigned to the CD-UID 314 to the SDoH request 304 for the individual 302, as shown by the letters E and F.

With reference again to FIG. 2, the SDoH scoring engine 206 uses the consumer marketing data loader 205 to obtain, from the consumer marketing database 108, consumer marketing data 316 on the individual 302. For example, the consumer marketing data loader 205 may generate a request that may specify that certain consumer marketing data elements are desired for “XYZ987;” wherein “XYZ987” is the CD-UID 314 associated with the individual 302. The consumer marketing database 108 may use the CD-UID 314 to retrieve the requested data elements and return the requested data elements to the consumer marketing data loader 205. In example aspects, the consumer marketing data elements requested by the consumer marketing data loader 205 are based on a plurality of socio-economic/environmental and/or personal/behavioral factors that the SDoH scoring engine 206 is configured to evaluate as part of determining a propensity score indicating a level of propensity of the individual incurring negative health impacts due to SDoH. For example, the consumer marketing data elements requested by the consumer marketing data loader 205 may correlate to particular indicators or factors associated with particular SDoH. Examples of particular factors and/or consumer marketing data elements requested by the consumer marketing data loader 205 include, but are not limited to: income, age, household composition, language preference, residential location, health-related business locations, job location, profitability related data, vehicle ownership, home value, etc. Other consumer marketing data elements may be requested by the consumer marketing data loader 205 in association with other factors that may be evaluated by the SDoH scoring engine 206 for determining an individual's propensity to incur negative health impacts due to SDoH.

In example aspects, when requested consumer marketing data elements are provided to the consumer marketing data loader 205 by the consumer marketing database 108, the data loader may be operable or configured to read, extract, and load consumer marketing data elements from a data file, such as a comma-separated values (CSV) file, or from a database connection. The consumer marketing data loader 205 may load the marketing data elements into a data store (e.g., the SDoH database 218) or may pass the marketing data elements to the SDoH scoring engine 206 as inputs into one or more algorithms used by the SDoH scoring engine 206 to obtain relevant business data and public transportation data and to calculate propensity scores for various factors that may affect the individual's propensity for negative health outcomes based on the marketing data, business data, and public transportation data. In some aspects, the consumer marketing data loader 205 may be further operable or configured to convert consumer marketing data elements that are received from the consumer marketing database 108 from the format in which they are received to a standardized format used by the SDoH scoring engine 206. For example, data fields in the received data file may be rearranged, truncated, dropped, relabeled, converted (e.g., from metric to standard units), combined (e.g., a first name field and a last name field merged into a name field), split (e.g., a name field broken into a first name field and a last name field), etc.

In some examples, the SDoH scoring engine 206 includes or is in communication with a location engine 213. The location engine 213 is operable or configured to access particular consumer marketing data elements from the received consumer marketing data for determining geographical criteria for a search for health-related businesses (e.g., healthcare facilities, pharmacies, therapy clinics), particular non health-related businesses (e.g., grocery stores), and public transportation stops/stations proximate to the individual's residence and/or work. In some examples, the location engine 213 may access and convert the individual's residential address and the individual's work address(es) into geolocation coordinates (e.g., latitude and longitude coordinates). The location engine 213 may use the addresses or the geolocation coordinates to determine a geographical area or radius in which a business search and public transportation search are to be performed. The searches, using the search criteria (i.e., geographical area or radius) may be conducted by the business data loader 209 and the public transportation data loader 211.

In some aspects, the business data loader 209 is configured to query or make a call to the business database 110 with a request for business information. The request may include the geolocation of the individual's residence and work locations and/or the radius or area within which to perform a search. The business database 110 returns relevant search results to the business data loader 209, which may include listings of health-related businesses and/or certain non-health-related businesses (e.g., grocery stores) in the area or within the search radius. The search results may further include the locations (e.g., addresses or geolocations) of the businesses. In some examples, the search may also return the distance between the business and the geographic location of the residence or work location (or otherwise specify its relative position with respect to the geographic location). Or, in other examples, the business data loader 209 provides the business locations to the location engine 213, which determines the distance between the business and the individual's residence or work location. In some examples, the business data loader 209 may convert an address into geolocation coordinates as part of determining the distance. In some examples, the search results may further include a category for each business located. For example, a doctor's office or pharmacy might return a category of “healthcare facility,” while a grocery store might return a category of “food” or “grocery.” The located businesses and location/distance data of the businesses may be stored in the SDoH database 218 and/or passed to SDoH scoring engine 206.

In some aspects, the public transportation data loader 211 is configured to query or make a call to the public transportation database 112 including a request for public transportation information, the geolocation of the individual's residence and work locations and, in some examples, a radius or area within which to perform a search. The public transportation database 112 returns relevant search results to the public transportation data loader 211, such as listings and locations of public transportation stops, stations, routes, etc., in the area or within the search radius. In some examples, the search may additionally return fare costs for using public transportation. In some examples, the search may also return the distance between the public transportation site (e.g., stop, station, route) and the geolocation of the residence or work location (or otherwise specify its relative position with respect to the geographic location). Or, in other examples, the public transportation data loader 211 provides the public transportation site locations to the location engine 213, which determines the distance between the public transportation site and the individual's residence or work location. In some examples, the public transportation data loader 211 may convert an address into geolocation coordinates as part of determining the distance. The search may further return a category for each public transportation site located (e.g., “bus,” “train,” “trolley”). The located public transportation sites and location/distance data of the public transportation sites may be stored in the SDoH database 218 and/or passed to SDoH scoring engine 206.

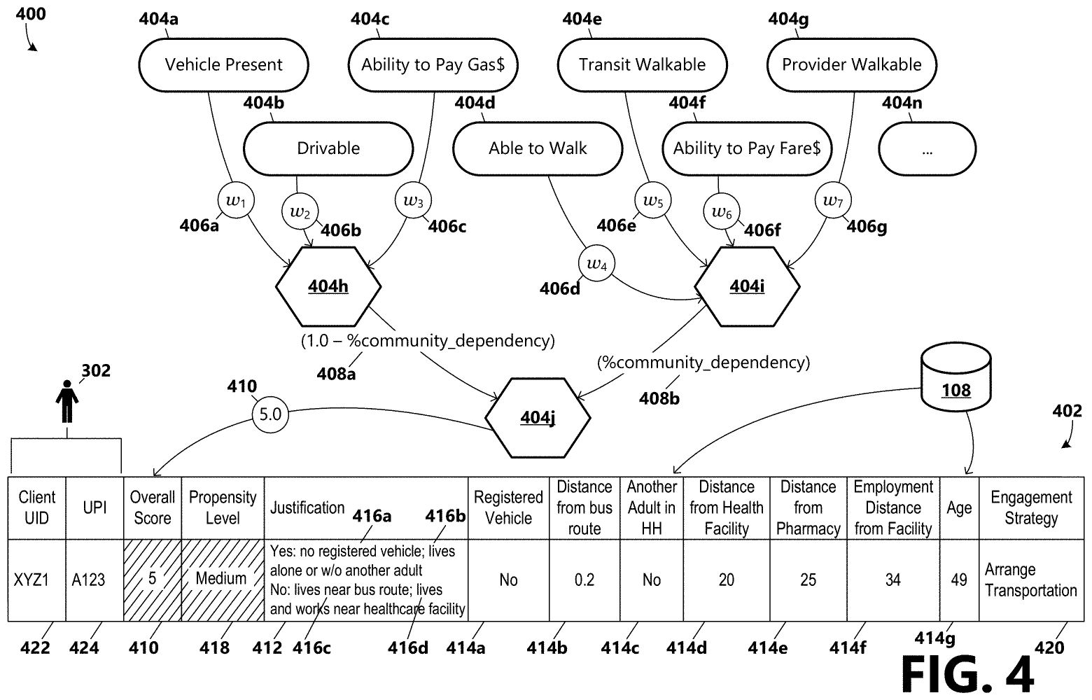

According to an aspect, the SDoH scoring engine 206 is operable or configured to use various collected data elements, including consumer marketing data elements that are received from the consumer marketing database 108, business data associated with health-related businesses and/or specific non health-related businesses proximate to the individual 302 and the locations of those businesses, and public transportation data associated with public transportation sites proximate to the individual and the locations of those public transportation sites, as inputs into one or more propensity models that evaluate the data for particular SDoH indicators or drivers (herein referred to as SDoH indicators) and compute a propensity score for each of various SDoH factors for which the SDoH scoring engine 206 is configured to evaluate. In some examples, the one or more propensity models compute SDoH indicator scores using a cumulative probability distribution function. Examples of various SDoH indicators that may be evaluated and scored for computing an SDoH factor propensity score include, but are not limited to: registered vehicles, distances of health-related businesses from the individual's residence and/or work, distances of health-related businesses from public transportation sites, distances of public transportation sites from the individual's residence and/or work, distance of grocery stores from the individual's residence and/or work, the individual's age, the individual's income, the individual's discretionary income, etc. Non-limiting examples of SDoH factors that the SDoH scoring engine 206 may compute SDoH factor propensity scores for and that may be computed into an overall SDoH propensity score may include, but are not limited to: access/inaccessibility to care, food insecurity, housing instability, interpersonal violence, social isolation, healthcare literacy, etc. SDoH indicator scores are explained in further detail below with respect to an example data flow illustrated in FIG. 4.

A computed SDoH factor propensity score may indicate an individual's level of propensity for a particular SDoH factor. As an example, an SDoH factor propensity score corresponding to inaccessibility to care may indicate the level of the individual's propensity for lack of access to a healthcare facility (e.g., whether the individual has or does not have access to/means for private transportation to a healthcare facility, whether the individual has or does not have access to/means for public transportation to a healthcare facility). For example, a high score may represent a high propensity for a lack of access, indicating that the individual 302 may not have a reliable means of transportation to a healthcare facility. Accordingly, a high inaccessibility to care SDoH factor propensity score may be used to identify inaccessibility to care as an SDoH that may have negative impacts to the individual's health.

As should be appreciated, in some implementations, a propensity/SDoH factor propensity score may correspond inversely to an SDoH factor. For example, if the SDoH factor being evaluated for an individual 302 is access to care (rather than inaccessibility to care), the SDoH factor propensity score may indicate the individual's propensity for access to care, wherein a higher score may indicate a higher propensity for the individual to have access to reliable access/transportation to care. That is, in some cases, a higher score corresponding to a particular SDoH factor (e.g., access to care) may indicate that that particular factor may not be an SDoH for which the individual is at risk. SDoH factor propensity scores are explained in further detail below with respect to the example data flow illustrated in FIG. 4.

According to an aspect, the SDoH scoring engine 206 is further operable or configured to determine an overall propensity score for an individual 302, wherein the overall propensity score may indicate an individual's level of propensity to incur negative health impacts due to one or more SDoH factors that are evaluated. The overall propensity score may be based on the scores of one or more evaluated SDoH factors (e.g., the scores of one or various SDoH factors may be factored into the computation of the overall propensity score), and various multipliers may be applied to each SDoH factor propensity score to vary the amount of impact each score can have on the overall propensity score. The individual's overall propensity score may indicate the individual's likelihood or propensity to incur negative health impacts due to the individual's access or lack of access to care in combination with other evaluated SDoH factors. For example, an overall propensity score may be used as a way of predicting whether an individual will have negative health impacts due to various SDoH factors associated with the individual. In various examples, the overall propensity score may be delivered as a value between 1-9, wherein a lower value indicates a lower propensity and a higher value indicates a higher propensity. Based on a calculated overall propensity score, the SDoH scoring engine 206 may determine an associated propensity level. For example, a score of 7-9 may be associated with a high propensity level, a score of 4-6 may be associated with a medium propensity level, and a score of 1-3 may be associated with a low propensity level. As should be appreciated, other score values and levels may be used. In some examples, the overall propensity score is computed using a machine-learned cumulative probability distribution function (e.g., pnorm(t(x),m(t(x)),sd(tx))). For example, training data, which may be comprised of historical data corresponding to demographic data, social data, and health outcome data for a corpus of individuals, may be analyzed by machine learning algorithms used to train models used by the SDoH scoring engine 206. The analysis of the training data may reveal relationships and trends between various social factors and actual health outcomes, and one or more models may be generated based on the analysis that represents these relationships and trends. Overall propensity scores are explained in further detail below with respect to the example data flow illustrated in FIG. 4.

With reference now to FIG. 4, a flow diagram of an example process 400 of evaluating consumer marketing data 316, business data, and public transportation data for generating an SDoH propensity solution 402 comprised of SDoH information about an individual is illustrated. As illustrated in FIG. 4, various propensity models 404a-n (generally 404) may evaluate various consumer marketing data 316, business data, and public transportation data for various SDoH indicators associated with an individual 302. For example, the various SDoH indicators being evaluated are associated with one or more SDoH factors for which the SDoH system 104 is determining an individual's 302 propensity. The propensity models 404 may be trained by machine-learning algorithms and used by the SDoH scoring engine 206 to compute a score 406 or value for each of various SDoH indicators associated with the particular SDoH factor(s) and an SDoH factor propensity score 408 from the combined indicator scores 406. Various multipliers may be applied to each SDoH indicator score 406 to vary the amount of impact each score can have on the SDoH factor propensity score 408.

In the illustrated example, a plurality of propensity models 404 may evaluate data for various SDoH indicators that may be associated with an individual's access or lack of access to healthcare. A first set of propensity models 404a-c,h may evaluate collected data for various SDoH indicators associated with a first SDoH factor: the individual's access or lack of access to healthcare using private transportation. Depending on the specific indicator being evaluated, the computation of an indicator score 406 may take into account the distance of the individual's residence and/or work from the geolocation of a healthcare facility, and can also consider other factors as well. For example, a first propensity model 404a may evaluate various data (e.g., consumer marketing data 316 associated with vehicles registered to the individual 302 or other individuals in the individual's household) for computing an indicator score 406a corresponding to the likelihood or probability of the individual having a vehicle present. For example, an indicator score 406 of 2.0 may be indicative of a low likelihood that the individual 302 has a vehicle, while an indicator score of 8.0 may indicate a high likelihood that the individual has a vehicle. A second propensity model 404b may evaluate various data (e.g., consumer marketing data 316 and/or demographic data 308 associated with the location of the individual's residence and/or location of the individual's work, business data associated with locations of healthcare facilities, data calculated from consumer marketing data, demographic data, and/or business data, such as: distances between the individual's residence and healthcare facilities and distances between the individual's work and healthcare facilities) for computing an indicator score 406b corresponding to the likelihood of a healthcare facility being drivable (e.g., based on distance). A third propensity model 404c may evaluate various data (e.g., consumer marketing data 316 associated with the individual's income or discretionary income) for computing an indicator score 406c corresponding to the likelihood of the individual's ability to pay for gas for driving to a healthcare facility.

According to an aspect, the computed indicator scores 406a-c for various SDoH indicators may be input into a fourth propensity model 404h that the SDoH scoring engine 206 may use for computing a first SDoH factor propensity score 408a corresponding to the individual's access or lack of access to healthcare using private transportation (the first SDoH factor). For example, indicator scores 406a-c, which are outputs of the first set of propensity models 404a-c, may be applied to a propensity model 404h that evaluates the indicator scores for determining the individual's likelihood to have access to private transportation to a healthcare. That is, the likelihood of the individual's access to private transportation to a healthcare facility (a first SDoH factor) may be represented as the first SDoH factor propensity score 408a that is based on the indicator scores 406a-c associated with the individual 302 having a vehicle present, how far healthcare facilities are from the individual (i.e., drivability), and whether the individual has discretionary income for gas money to drive a private vehicle to the healthcare facility. Various multipliers may be used to vary the amount of impact each indicator can have on the first SDoH factor propensity score 408a.

Continuing with the illustrated example, a second set of propensity models 404d-g,i may evaluate collected data for various SDoH indicators associated with a second SDoH factor: the individual's access or lack of access to healthcare using public transportation. For example, a fifth propensity model 404d may evaluate various data (e.g., consumer marketing data 316, demographic data) for computing an indicator score 406d corresponding to the likelihood of the individual's ability to walk. A sixth propensity model 404e may evaluate various data (e.g., consumer marketing data 316 and/or demographic data 308 associated with the location of the individual's residence and/or location of the individual's work, business data associated with locations of healthcare facilities, public transportation data associated with locations of public transportation sites) for computing an indicator score 406e corresponding to the likelihood of walkability to public transportation. A seventh propensity model 404f may evaluate various data (e.g., consumer marketing data 316 associated with the individual's income or discretionary income) for computing an indicator score 406f corresponding to the likelihood of whether the individual has discretionary income for money to pay for transit fares to a healthcare facility. An eighth propensity model 404g may evaluate various data (e.g., consumer marketing data 316 and/or demographic data 308 associated with the location of the individual's residence and/or location of the individual's work, business data associated with locations of healthcare facilities) for computing an indicator score 406g corresponding to the likelihood of the individual's ability to walk a healthcare facility.

According to an aspect, the computed indicator scores 406d-g for various SDoH indicators may be input into a ninth propensity mode 404i that the SDoH scoring engine 206 may use for computing a second SDoH factor propensity score 408b corresponding to the individual's dependency on his/her community for access to healthcare using public transportation (the second SDoH factor). That is, the likelihood of the individual's access to public transportation to a healthcare facility may be represented as an SDoH factor propensity score 408b that is based on the indicator scores 406d-g associated with the individual's ability to walk, whether transit is walkable, whether the individual 302 has discretionary income for transit fare money to the healthcare facility, and how far healthcare facilities are from the individual (i.e., walkability). Various multipliers may be used to vary the amount of impact each indicator can have on the second SDoH factor propensity score 408b.

Continuing with the illustrated example, an overall propensity score 410 for the individual 302 may be computed by a tenth propensity model 404j used by the SDoH scoring engine 206 based on the outputs (i.e., SDoH factor propensity scores 408a,b) of the eighth and ninth propensity models 404h,i. For example, the overall propensity score 410 may estimate the likelihood or propensity that the individual may not have access to healthcare, wherein a low overall propensity score may indicate a low likelihood that access to healthcare is an SDoH factor for the individual 302, and a high overall propensity score may indicate a high likelihood that access to healthcare is an SDoH factor for the individual. With continued reference to FIG. 4, the overall propensity score 410 in the illustrated example may be based on one or more SDoH factor propensity scores 408 corresponding to private transportation and public transportation access, wherein the private and public transportation access SDoH factor propensity scores 408 may be based on one or more SDoH indicators (e.g., registered vehicles, distance to public transportation, household makeup, distance to a healthcare facility, distance to a pharmacy, distance from workplace to a healthcare facility, age) that are learned about the individual 302 from the collected data.

As mentioned above, various multipliers may be used to vary the amount of impact each SDoH factor propensity score 408a,b can have on the overall propensity score 410. As should be appreciated, fewer, additional, and/or alternative propensity models 404 may be trained and used to evaluate various consumer marketing data 316, business data, public transportation data, and/or other data for identifying or determining various SDoH indicators and SDoH factors that may be associated with an individual's health outcome. Examples of other SDoH factors that may be taken into account in the calculation of an overall propensity score 410 may include, but are not limited to: food insecurity, housing instability, interpersonal violence, social isolation, and healthcare literacy. As an illustrative example, consumer marketing data 316 may include data associated with an amount spent by an individual 302 (and/or the individual's household) on packaged meals. This data may be used as an input into one or more propensity models 404 that compute an SDoH factor propensity score 408 associated with a food insecurity SDoH factor. For example, a high food insecurity SDoH factor propensity score may be indicative of a possible food insecurity issue that may contribute negatively to the individual's health outcome.

According to an aspect, outputs of the SDoH scoring engine 206 may be stored in the SDoH database 218 and/or may be passed to the justification engine 220. The justification engine 220 is operable or configured to determine a justification 412 and justification details 414a-n (generally 414) for a computed SDoH factor propensity score 408 or an overall propensity score 410 that provide insight into factors that drive the propensity score. As will be described in further detail below, the justification 412 and justification details 414 may be included, by the report generator 212, in an SDoH propensity solution 402 that is provided to the client device 102 in response to an SDoH request 304. The justification 412 and justification details 414 may correspond with various SDoH indicators of the evaluated SDoH factors on which SDoH factor propensity scores 408 and the overall propensity score 410 are based. As an example, if access to care is an SDoH factor that is being evaluated, the justification details 414 may include actual data, the calculated scores, and/or binary results for the SDoH indicators that are included in the computation of the access to care SDoH factor propensity score. For example, for an SDoH indicator corresponding to registered vehicles associated with an individual 302, the justification detail 414a for the SDoH indicator may be a binary result of “no,” indicating that the individual or a person in the individual's household does not have a registered vehicle, or may be a value, such as “0.2,” that indicates the likelihood of the individual/individual's household having a registered vehicle. As another example, for an SDoH indicator corresponding to the distance between an individual's residence and public transportation, the justification detail 414b for the SDoH indicator may be a corresponding SDoH indicator score 406e (e.g., 0.1, 5.0, 18, or another value corresponding to the distance between the individual's residence and a public transportation route, stop, or station). As another example, for an SDoH indicator corresponding to the individual's age, the justification detail 414g for the SDoH indicator may be the individual's age (i.e., actual data collected from the demographic database 106, the consumer marketing database 108, the business database 110, the public transportation database 112, or another database 114). The justification details 414, when included in an SDoH propensity solution 402 provided to the client for display on a display of the client device 102, communicate useful information and insights from the collected data and provide context for an SDoH factor propensity score 408 and/or overall propensity score 410 determined for an individual 302.

A justification 412 may be a human-readable output that describes one or more justification details 414. A justification 412 for an overall propensity score 410 may be comprised of a plurality of justifiers 416a-n (generally 416) that describe each or a plurality of SDoH indicators factored into an SDoH factor propensity score 408. That is, a justifier 416 may be used to communicate whether data evaluated for an indicator of an SDoH factor may support or counter an individual's propensity for the SDoH factor.

In some examples, the justification engine 220 may be configured to analyze an SDoH indicator score 406 for an SDoH indicator in relation to the SDoH factor propensity score 408 or overall propensity score 410 for determining the effect of the indicator on the factor or overall score. For example, the justification engine 220 may analyze the outputs of the SDoH scoring engine 206 (e.g., SDoH indicator scores 406, SDoH factor propensity scores 408, overall propensity score 410) for determining the effect of each SDoH indicator on the SDoH factor propensity score 408 and/or overall propensity score 410. In some examples, the effect may be determined based on whether the SDoH indicator score 406 increments or decrements the SDoH factor propensity score 408 or overall propensity score 410. An SDoH indicator score 406 that increments the SDoH factor propensity score 408 or overall propensity score 410 may be associated with an indicator of an SDoH factor that supports an individual's propensity for the SDoH factor that may increase the likelihood of the individual incurring negative health impacts. An SDoH indicator score 406 that decrements the SDoH factor propensity score 408 or overall propensity score 410 may be associated with an indicator of an SDoH factor that counters an individual's propensity for the SDoH factor, and thus may not increase the likelihood the individual incurring negative health impacts.

The justification engine 220 may be communicatively connected to a justification rules database 222 that stores justification rules for determining a justifier 416 for an SDoH indicator. For example, the justification engine 220 may be configured to evaluate the SDoH indicator scores 406 against a set of justification rules for determining justifiers 416 that correspond to the driving indicators of the SDoH factor. The justification engine 220 may perform a lookup operation of the justification rules database 222 for a rule that applies to each SDoH indicator or to each of a subset (e.g., SDoH indicators with the highest impact on the factor or overall score) of SDoH indicators for determining a justifier 416 for the indicator. As an example, consider an SDoH indicator score 406 associated with an individual's distance from a public transportation route that is computed as a 0.2. The justification engine 220 may be configured to look up a rule that applies to the SDoH indicator score 406 (or to a range of scores which the SDoH indicator score 406 may fall within). For example, a rule stored in the justification rules database 222 may be used to apply a particular justifier 416 for an SDoH indicator with a score 406 that falls within a range of 0.0-1.0.

In some example aspects, the justifier 416 may communicate whether the SDoH indicator supports an SDoH factor or counters the SDoH factor. For example, a justifier 416 for the example SDoH indicator score 406 may include text such as, “no: lives near public transportation,” indicating that the individual's distance from public transportation may not negatively affect the individual's access to healthcare. In some examples, the justification engine 220 may use actual data associated with the individual to include in a justifier 416. For example, if a bus route is identified as a public transportation option that the individual lives near, the corresponding justifier 416c may include text such as, “lives near a bus route.” As another example, the justification engine 220 may use actual distance data to include in the justifier 416, such as, “lives <0.2 miles from a bus route.” As should be appreciated, these examples are meant to be illustrative and not limiting; alternative and additional phrasings may be used. According to an aspect, justifiers 416 in a justification 412 may be grouped based on whether the corresponding SDoH indicator supports or counters the SDoH factor (e.g., justifiers of indicators that support propensity for the SDoH factor may be grouped together and justifiers of indicators that counter propensity for the SDoH factor may be grouped together).

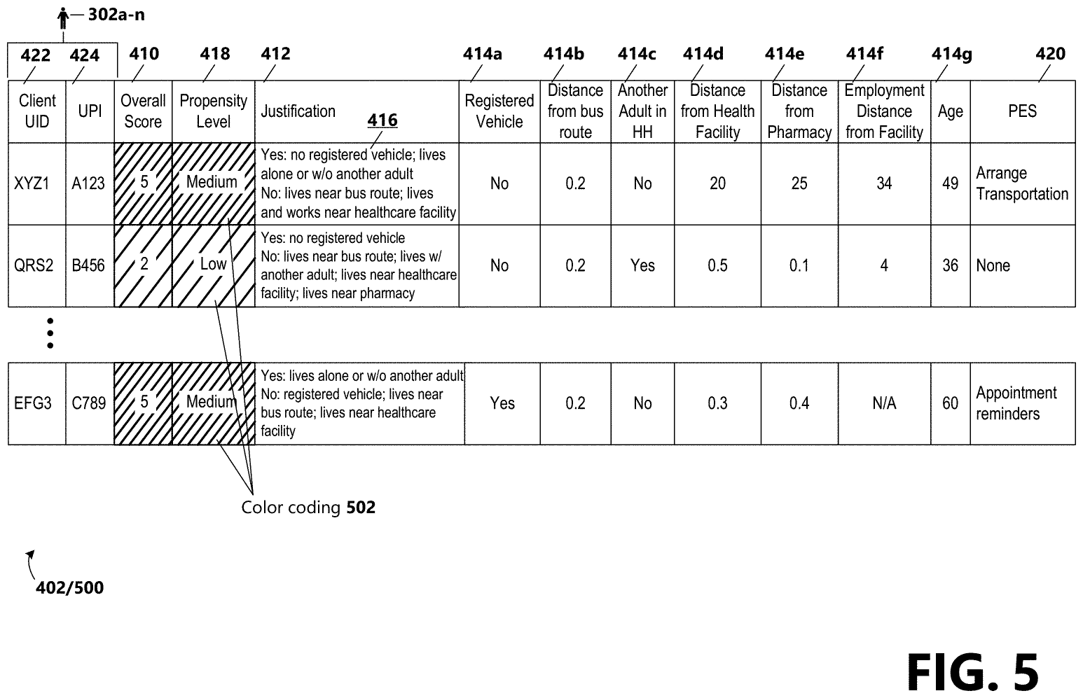

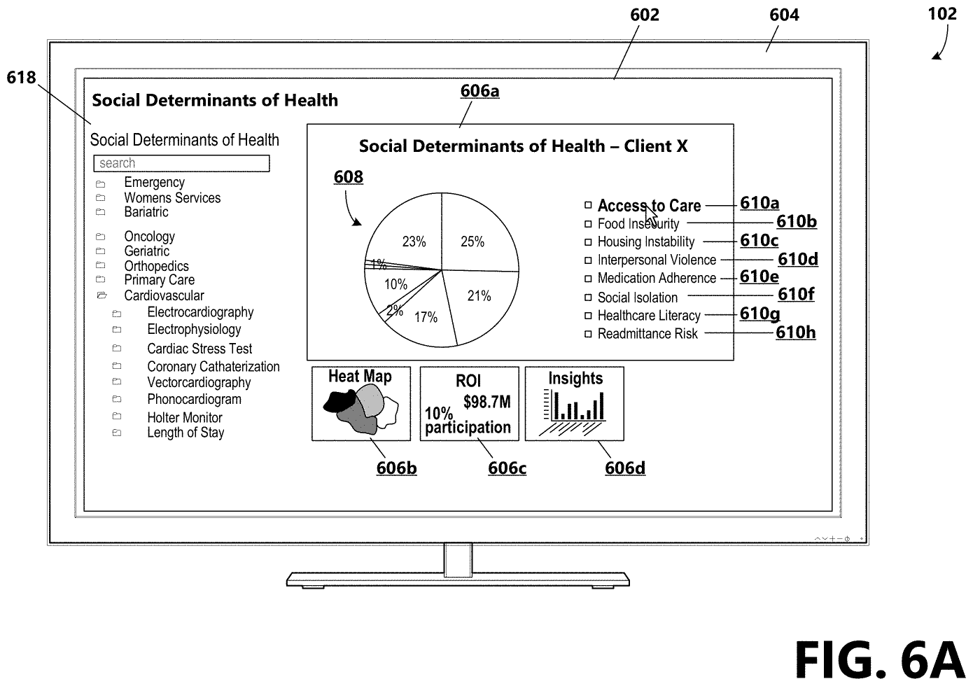

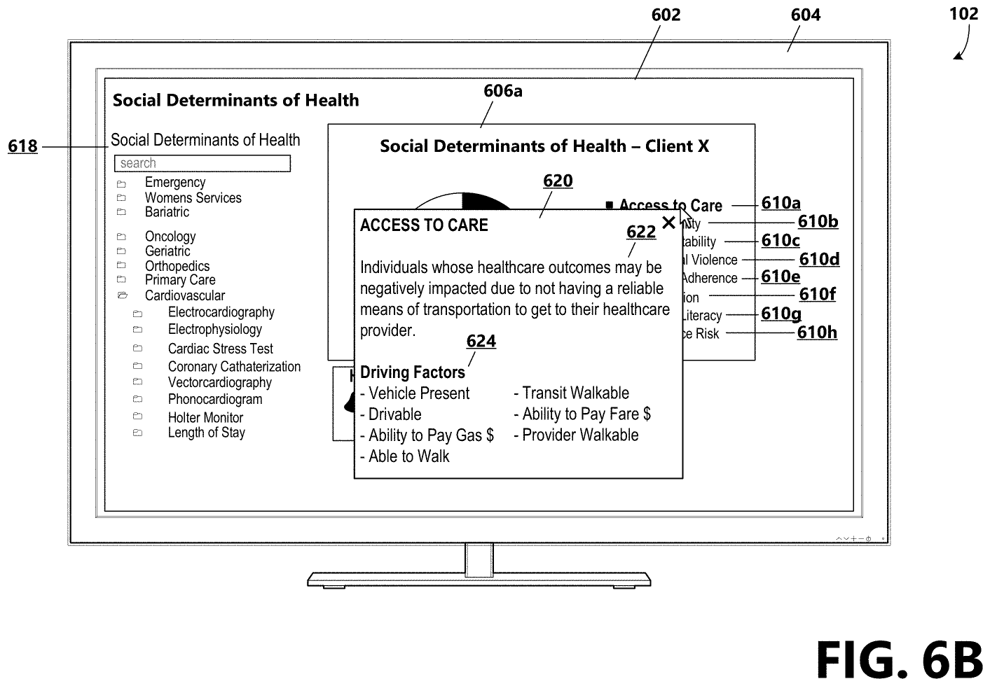

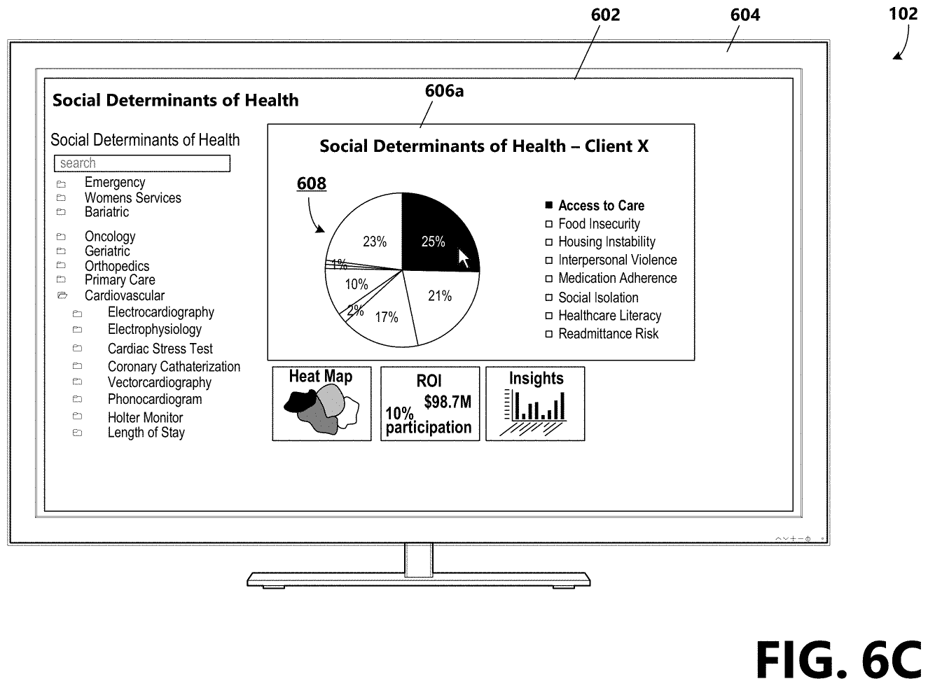

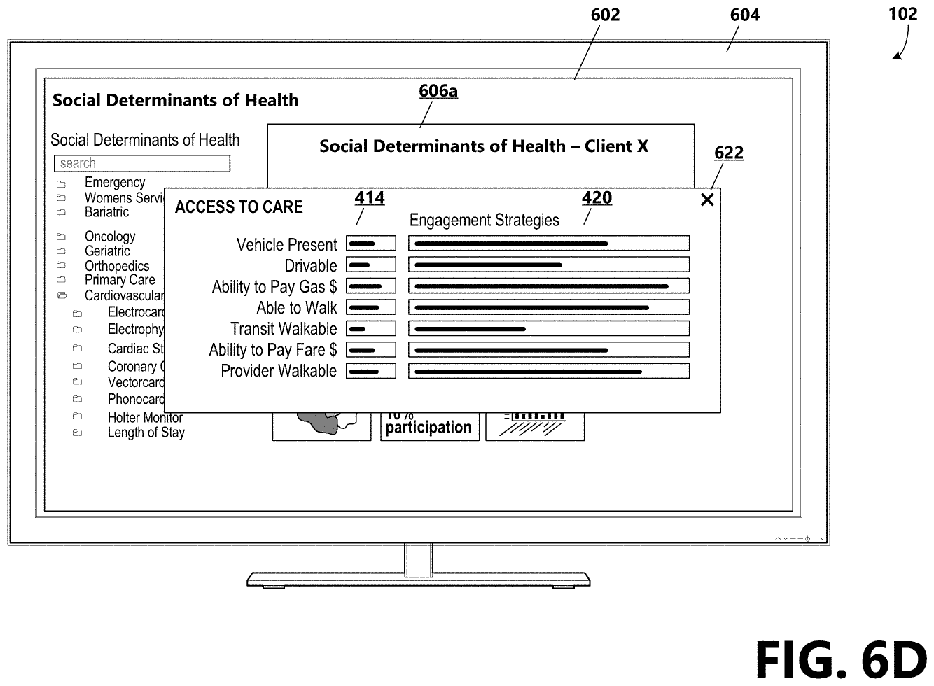

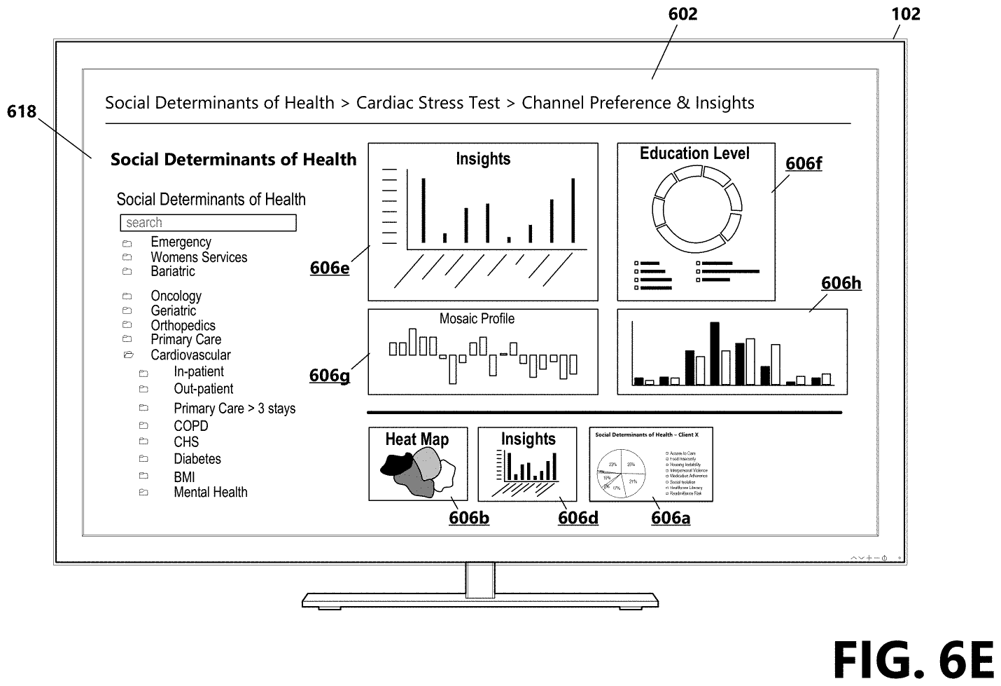

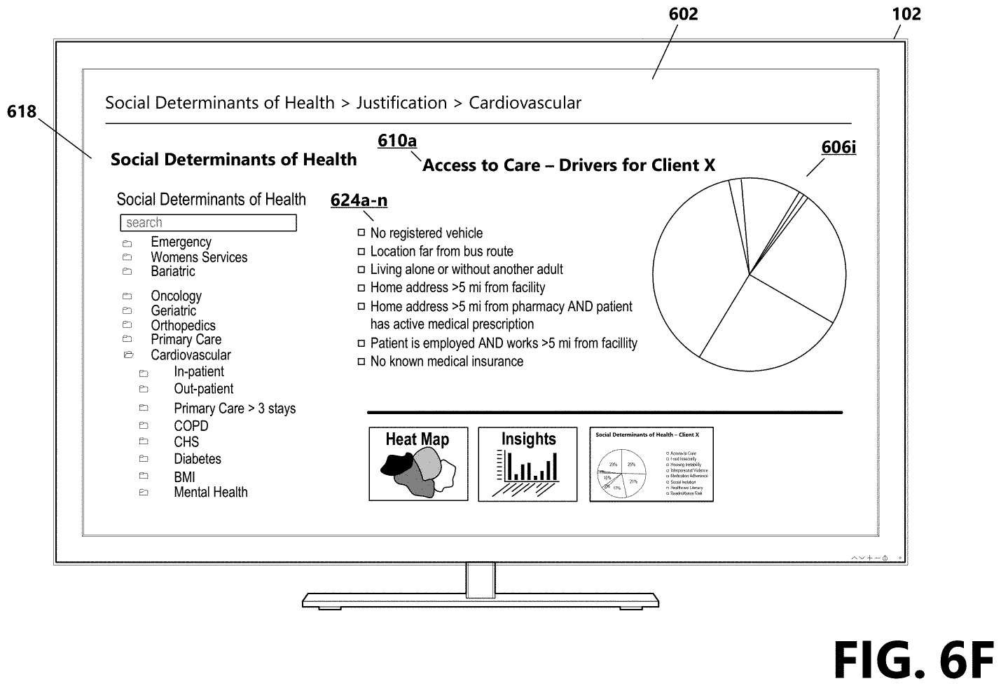

In the example SDoH propensity solution 402 results illustrated in FIG. 4, the justification 412 includes the justifiers 416a,b “no registered vehicle” (416a) and “lives alone or without another adult” (416b) in support of the individual's propensity for the SDoH factor of inaccessibility to healthcare, and further includes the justifiers 416c,d “lives near bus route” (416c) and “lives and works near healthcare facility (416d) that are grouped as justifiers that may not be in support of the SDoH factor. As will be described in further detail below, in some examples, the justification 412 and justification details 414 may be included, by the report generator 212, in a report or as part of an SDoH propensity solution 402 that is provided to the client device 102 in response to an SDoH request 304. In other example aspects, the SDoH propensity solution 402, which may include the justification 412 and justification details 414, may be included, by the user interface engine 214, in a user interface generated for display on the client device 102. SDoH propensity solution 402 results are explained in further detail below with respect to example SDoH propensity solution results illustrated in FIGS. 5 and 6A-C.

According to an aspect, the SDoH propensity solution 402 may further include an engagement strategy 420 for the individual 302 determined by the engagement strategy engine 208, wherein the engagement strategy may be an effective reach-out or engagement approach that has been determined to be an effective means of addressing an SDoH factor need and positively impacting an individual's health outcome(s). Non-limiting examples of engagement strategies 420 for the SDoH factor of inaccessibility to healthcare may include recommended actions such as: arrange transportation for the individual, provide remote healthcare services for the individual (e.g., telehealth consultation), provide appointment reminders for the individual, provide the individual a bus/train/other public transportation pass, arrange a shuttle pickup, etc. As can be appreciated, various engagement strategies 420 may be determined and provided to a client for addressing various types of SDoH factors, such as: housing instability, food insecurity, low access to healthful or affordable food, having emotional or mental stress caused by financial instability, etc.

According to an aspect, the engagement strategy engine 208 may be configured as a rules engine in communication with an engagement strategies rules database 210, wherein the database stores engagement strategies and rules associated with the engagement strategies 420. For example, the rules may define criteria (i.e., conditions) that determine if and when a particular engagement strategy 420 is relevant to an individual 302 for addressing one or more social factors corresponding to the individual's propensity for an SDoH. The engagement strategy engine 208 may evaluate various data (e.g., overall propensity score 410, justification 412, justification details 414) for determining if certain rule criteria are met for determining which engagement strategy 420 or strategies apply. As one of ordinary skill in the art will appreciate, engagement strategies rules may be implemented via individual transistors that are discrete components of a circuit or via a processor that is configured by software to provide the corresponding logical operations necessary for comparison. In some implementations, an engagement strategy rule may be defined that causes the engagement strategy engine 208 to determine and provide one or more engagement strategies 420 for addressing the one or more SDoH that may have the largest impact on the individual's overall propensity score 410 (e.g., based on the evaluation of the overall propensity score 410, justification 412, and justification details 414).

A particular engagement strategy 420 may be determined as relevant to an individual 302 based on one or more sets of rule criteria. In some examples, the rule criteria may be based on specific details in the justification 412 and the justification details 414). When criteria associated with an engagement strategy 420 are met, the engagement strategy engine 208 may apply the associated engagement strategy to the SDoH propensity solution 402 for the individual. As an example, an engagement strategy rule may define an engagement strategy of “arranging transportation” for an individual 302 when certain criteria associated with the individual's lack of access to healthcare (i.e., SDoH factor) are met. The determination of whether the rule criteria are met may be based on an analysis of the justification 412 and justification details 414 associated with a computed overall propensity score 410 and/or propensity level 418. For example, one set of rule criteria associated with the “arranging transportation” engagement strategy 420 may include a combination of criteria such as: no registered vehicle (justifier 416a) and lives alone or without another adult (justifier 416b).

According to an example, in response to an SDoH request 304, the report generator 212 may include the engagement strategy 420 in a report or as part of an SDoH propensity solution 402 that is provided to the client device 102. In other example aspects, an SDoH propensity solution 402 including the engagement strategy 420, may be included, by the user interface engine 214, in a user interface generated for display on the client device 102. As can be appreciated, by providing the engagement strategy 420 to the client for display to client users, client organizations are enabled to act on identified SDoH factors using a recommended and effective engagement strategy. For example, a client user may be enabled to react in real time to an individual 302 based on an engagement strategy 420 presented to the user.

As an example, the individual 302 may be a patient being discharged from a healthcare facility and who may be identified as an individual who is at risk (i.e., medium or high propensity) for an SDoH factor that may be resolvable by applying the recommended engagement strategy 420 that is determined to be relevant to the individual 302 based on an evaluation of information about the individual. The client user may act on the recommended engagement strategy 420 to schedule resources (e.g., transportation, remote services), provide resources (e.g., public transportation pass), or enroll the individual 302 in a community program that addresses the individual's SDoH needs. Providing the client with an appropriate patient engagement strategy 420 can help clients be more patient-centered because the engagement strategy informs the client about how to engage with the individual 302 to reduce the likelihood for negative health outcomes in the form of readmissions, no-shows, emergency department usage, etc.

The report generator 212 is operable or configured to generate an output including the SDoH propensity solution 402 results determined for one or more individuals 302, wherein the output and SDoH propensity solution results are configured for transmission to a client device 102 and for display on the client device. For example, the SDoH propensity solution 402 may include one or more individuals' overall propensity score 410 and propensity level 418, the justification 412 and justification details 414 for the overall propensity score, and one or more engagement strategies 420 that identifies, informs, and provides a solution for addressing the one or more individuals' SDoH. The form of the output and the delivery options may vary based on different software systems and/or client types. For example, SDoH propensity solution 402 results may be provided via a batch output or may be provided in a response to an API call made by a client device 102 to the SDoH system 104, wherein the API call may include a real time or near-real time request for SDoH propensity information for an individual 302.

With reference now to FIG. 5, an illustration is provided that includes example SDoH propensity solution 402 results provided in a batch file 500. For example, a client, via a client device 102, may send a batch file 500 to the SDoH system 104 as part of an SDoH request 304, wherein the batch file may be in the form of a table or spreadsheet that includes a listing of individuals 302 about which the client is seeking SDoH information. Each individual 302a-n included in the batch file 500 may be identified by identifying data 306, such as a client unique identifier 502 and/or demographic data (e.g., name, address, date of birth). Responsive to receiving the batch file 500/SDoH request 304, the SDoH system 104 may determine an SDoH propensity solution 402 for each individual 302 included in the batch file, insert the SDoH propensity solution results in the batch file (e.g., insert one or more columns into the table or spreadsheet that include the determined overall propensity score 410, the propensity level 418, justification 412, justification details 414, and engagement strategy 420 for each individual), and transmit the batch file 500 including the solution results to the client.

According to an aspect, the report generator 212 may be further operable or configured to apply color coding 502 to the output/SDoH propensity solution 402 results. For example, a high overall propensity score 410 or a high propensity level 418 may be shaded red, a medium propensity score or a medium level may be shaded yellow, and a low propensity score or a low level may be shaded green. As should be appreciated, other colors or color coding 502 may be used. The color coding 502 may be applied to the text of the propensity score and/or level, or the color coding may be applied to the cell of the table or spreadsheet that includes the propensity score and/or level. In other examples, the color coding 502 may be provided as an alternative to the propensity score and/or level (e.g., a color code is provided that is indicative of the propensity score/level). According to aspects, the color coding 502 enables a client device user 102 to identify and act on an individual's SDoH easier than an ambiguous score. When provided in a batch 500, the color coding 502 allows client users to interpret batch results more quickly and to be able to triage and work patients based on priority corresponding to their propensity or other specific SDoH factors.

In some examples and with reference to FIGS. 6A-F, the report generator 212 may use a user interface (UI) engine 214 to create a user-navigable UI 602 that can be transmitted to a client device 102 for enabling a client to view output/SDoH propensity solution 402 results on a display of the client device 102. For example, the SDoH system 104 may provide an API 216 that a platform or software application executing on the client device 102 may communicate with for accessing SDoH propensity solution 402 results. That is, the platform or software application executing on the client device 102 may be configured to call the API 216, request and receive SDoH information, and process received SDoH propensity solution results data for displaying the results in the platform or software application. In some examples, a platform or software application executing on the client device 102 may call the API 216 for an individual lookup and display of SDoH propensity solution 402 results data. In other examples, a platform or software application executing on the client device 102 may call the API 216 for lookup and display of aggregate levels of SDoH propensities for various segments of patients or the population. Examples of displays of aggregate levels of SDoH propensities for various segments of patients or the population are illustrated in FIGS. 6A-F.

With reference now to FIG. 6A, an example graphic UI (GUI) 602, which is illustrative of a GUI that may be generated by the UI engine 214, is shown displayed on a display 604 of a client device 102. The example GUI 602 includes a plurality of visualizations 606a-n (generally 606) of SDoH results data, wherein the visualizations may be representative of SDoH propensity solution 402 results determined for a single individual 302, such as a patient of a healthcare service provider (i.e., client), or may be representative of a population or group of individuals, such as a group segmented by service provider/client, by service provider type, by patient type, by demographic indicator(s), by location, or by another indicator. According to the illustrated example, a first visualization 606a may include a pie chart 608 that represents a numerical proportion of various SDoH factors 610a-h that may affect a population, such as a client's patient base, individuals within a geographic location, certain patient types, or other category of individual. Other visualizations that may be selectively displayed in the GUI 602 may include a heat map 612 representative of SDoH information about a geographic segment of a population, return on investment (ROI) and patient participation data 614, a chart representative of other insights 616 associated with SDoH propensity solution results (e.g., justification details 414, information about non-clinical factors that impact healthcare outcomes. According to some aspects, the GUI 602 may include a listing of segments 618 from which a segment can be selected for viewing data associated with that segment. For example, segments 618 may be client-based, geography-based, practice area-based, demographically-based, etc. In the illustrated example, the listing of segments 618 includes a listing of healthcare practice areas, which may be categorized further into particular procedures, services, equipment, or other healthcare service indicators. In some examples, the listing of segments 618 includes a search box that allows the client user to enter a keyword or phrase (query) and submit it to search an index of segments for a segment into which SDoH data can be classified and represented in one or more GUI visualizations 606.

In some examples and as illustrated in FIG. 6B, in response to a particular interface interaction (e.g., hover, selection) in association an SDoH factor 610a displayed in the GUI 602, the GUI may be updated to display a UI element 620 (e.g., a message box, a notification, a tooltip) that includes information about the corresponding SDoH factor 610a. For example, the UI element 620 may include a definition 622 of the SDoH factor 610a, driving factors 624 or indicators of the SDoH factor, and/or other information associated with the SDoH factor. If the client user hovers over another SDoH factor 610b-h displayed in the GUI 602, the GUI may be updated again to display information about the other SDoH factor in the same or another UI element 620.

In some examples and as illustrated in FIG. 6C, the client user may perform another particular interface interaction (e.g., hover, selection) in association an SDoH factor 610a represented in the visualization 606 (e.g., pie chart 608) in the GUI 602. Responsive to the other particular interface interaction, and with reference now to FIG. 6D, the example GUI 602 may be updated to display another UI element 622 (e.g., a message box, a notification, a tooltip) that includes information about the corresponding SDoH factor 610a, such as justification details 414 and/or engagement strategies 420. FIG. 6E includes another example GUI 602 including a dashboard interface that includes a plurality of other data visualizations 606 representative of various SDoH propensity solution results data.

FIG. 6F includes another example GUI 602, wherein the example GUI includes a data visualization 606i representative of various SDoH factor drivers or indicators 624a-n (generally 624) that are determined to affect the client's patient base based on the SDoH propensity solution 402 generated by the SDoH system 104. For example, the data visualization 606i may provide the client user with insights into the client's patient base for enabling the client to act on the insights for addressing one or more SDoH factors (generally 610) for improving health outcomes of the patients. As an example, if the data visualization 606i indicates that a sizable portion of the client's patient base does not have reliable means of transportation to healthcare (i.e., has a propensity to lack of access to care), the client may use this data to determine an appropriate patient care plan or engagement strategy. The example GUIs 602 illustrated in FIGS. 6A-F are not meant to be limiting of the various GUIs that may be generated by the UI engine 214 and provided to the client device 102 for displaying SDoH propensity solution 402 results.

According to an aspect, SDoH propensity solution 402 results displayed in a batch 500 report or in a GUI 602 may provide a drill-down into leading indicators and engagement strategies 420 that make understanding the social determinants that impact healthcare populations more actionable at more levels of an organization. For example, a care manager user may view an SDoH propensity solution 402 comprised of individual look up results and an SDoH profile of an individual, and use the information in the solution to enroll an individual 302 in a care plan or to schedule a care plan accommodation to address the individual's SDoH through the organization's available programs or community programs. As another example, an executive director user may view aggregate profiles of SDoH across populations segmented by the organization's own clinical data or geography-based segments to understand the best areas or populations for population health investment initiatives.

As can be appreciated, SDoH propensity solution 402 results generated and provided by the SDoH system 104 can save healthcare organizations dollars on blanket-approach programs that do not utilize data that define “who” needs/can benefit from the programs. Additionally, generating and providing SDoH propensity solution 402 results to a healthcare organization can save the organization dollars by preventing potential no-shows due to SDoH. Examples of costs associated with no-shows include lost opportunity to use medical equipment reserved for the patient's appointment time, lost opportunity of medical professionals' time, etc. Further, SDoH propensity solution 402 results can provide cost relief to healthcare organizations and patients from excessive healthcare utilization. For example, patients who call an ambulance as their mode of healthcare transportation or who return to the hospital frequently because health conditions may not have been under control due to a lack of ability to comply with recommended care plans (e.g., picking up prescription medication) due to socio-economic factors. By providing SDoH propensity solution 402 results as a tool to clients, clients can use the results data to address various socio-economic factors that may cause certain patients to have an unequal ability to comply with recommended care plans, which can help prevent ailments or illnesses.

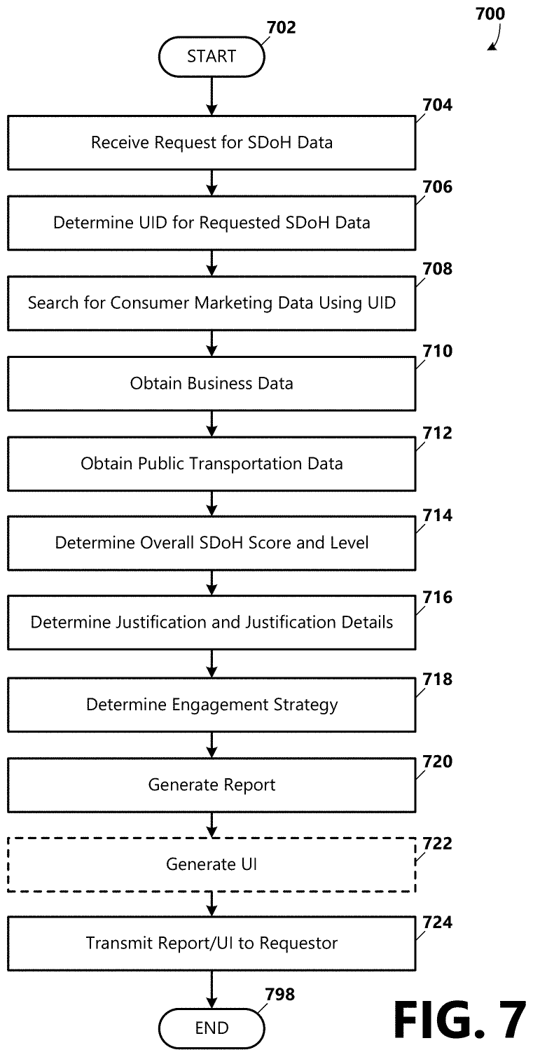

FIG. 7 illustrates a flow chart showing general stages involved in an example method 700 for generating and providing an SDoH propensity solution 402 to a requestor. The method 700 begins at START OPERATION 702 and proceeds to OPERATION 704, where the method 700 uses the receiver component 202 to receive a request 304 for SDoH information for one or more individuals 302. In some implementations, the request 304 may specify one or more types of SDoH factors for which the requestor desires information. In example aspects, the one or more individuals 302 may be identified by identifying data 306 (e.g., demographic data, client UID, other identifying) included in the SDoH request 304. In some examples, the method 700 uses the receiver component 202 to extract specific identifying data 306 from the SDoH request 304 and pass the extracted data to the identifier manager 204. In some examples, the method 700 may further use the receiver component 202 to convert identifying data 306 that are received in the SDoH request from the format in which they are received to a standardized format used by the identifier manager 204.

At OPERATION 706, the method 700 may use the identifier manager 204 to match the identifying data 306 associated with the one or more individuals included in the SDoH request 304 to demographic data 308 and a UID corresponding to each of the one or more individuals 302. For example, demographic data in the identifying data 306 may be used by the identifier manager 204 to correlate an individual to demographic data 308 stored in the demographic database 106 and to a first UID (UPI 310) that can then be mapped to a second UID (CD-UID 314) that can be used to obtain, from the consumer marketing database 108, consumer marketing data corresponding to the individual. In various examples, the first UID (UPI 310) may be a universal identifier that is used to link various data records from a plurality of data sources that correspond to the individual 302. In various examples, UIDs (e.g., UPI 310, CD-UID 314, or other UIDs) for individuals are stored in the UID database 107, which the identifier manager 204 may use for mapping the first UID to the second UID.

At OPERATION 708, the method 700 may use the consumer marketing data loader 205 to retrieve consumer marketing data 316 on the one or more individuals 302 from the consumer marketing database 108 using each individual's CD-UID 314. In example aspects, specific consumer marketing data elements corresponding to particular socio-economic/environmental and/or personal/behavioral factors associated with one or more SDoH are retrieved from the consumer marketing database 108. Examples of consumer marketing data elements retrieved by the consumer marketing data loader 205 that may be evaluated for determining an individual's propensity for an SDoH include, but are not limited to: income, age, household composition, language preference, residential location, health-related business locations, job location, profitability related data, vehicle ownership, home value, etc. The retrieved consumer marketing data 316 may be stored in the SDoH database 218 and/or passed to the SDoH scoring engine 206.

At OPERATION 710, the method 700 may use the business data loader 209 to obtain, from the business database 110, business data on health-related businesses (e.g., hospitals, doctors offices, health clinics, pharmacies, therapy centers) and/or non-health-related businesses (e.g., grocery stores) within a search area of the individuals' home and/or work locations. The business data obtained from the business database 110 may include the types of businesses (e.g., grocery store, pharmacy, type of healthcare practice) and the locations and/or addresses of the businesses, and may further include additional business-related information such as hours, types of insurance accepted, and other information that can be evaluated as part of determining a likelihood of an individual accessing/utilizing the business. The business data obtained from the business database 110 may be stored in the SDoH database 218 and/or passed to SDoH scoring engine 206.

In some implementations, the method 700 may use the data loader system 116 to obtain additional data associated with the one or more individuals 302 from other databases 114. For example, the data loader system 116 may access health-related information that may be held and stored by various originating and aggregating databases, such as but not limited to: patient type (e.g., outpatient, inpatient, emergency department, urgent care), healthcare coverage information (e.g., payer information), healthcare providers involved in the user's care, information associated with diagnoses, medications, family medical history, lab and test results, biometric data, treatment history, etc. Other types of user data are possible and are within the scope of the present disclosure.

At OPERATION 712, the method 700 may use the public transportation data loader 211 to obtain, from the public transportation database 112, public transportation data, such as listings and locations of public transportation stops, stations, routes, etc., within a search area of the individuals' home and/or work locations. The public transportation data obtained from the public transportation database 112 may include additional information, such as fare costs, hours, etc. Public transportation data obtained from the public transportation database 112 may be stored in the SDoH database 218 and/or passed to SDoH scoring engine 206.

At OPERATION 714, the method 700 may use the SDoH scoring engine 206 to determine an overall propensity score 410 and propensity level 418 for each individual 302. For example, the SDoH scoring engine 206 may use one or more propensity models 404 to evaluate the collected consumer marketing data 316, business data, public transportation data, and other data to compute SDoH indicator scores 406 for various indicators associated with one or more SDoH factors (e.g., access to care, food insecurity, housing instability), SDoH factor propensity scores 408 for the one or more SDoH factors, and an overall propensity score 410 that may indicate each individual's propensity to incur negative health impacts due to the one or more SDoH factors. Based on the computed overall propensity score 410, the SDoH scoring engine 206 may determine a propensity level 418 corresponding to the score, wherein the overall propensity score 410 and level 418 may indicate the individual's propensity for a particular SDoH factor or for a plurality of SDoH factors. For example, an overall propensity score 410 between 1-3 may be determined as a high propensity level, a score between 4-6 may be determined as a medium propensity level, and a score between 7-10 may be determined as a high propensity level. The overall propensity scores 410 and propensity levels 418 may be stored in the SDoH database 218 and/or passed to the justification engine 220.

At OPERATION 716, the method 700 may use the justification engine 220 to determine a justification 412 and justification details 414 for each SDoH factor propensity score 408 or overall propensity score 410, wherein the justification and justification details provide insight into factors that drive the propensity score. According to an aspect, the justification engine 220 may analyze the indicators that are associated with an SDoH factor and the scores corresponding to those indicators to determine how the indicators may drive or affect the SDoH factor propensity score 408. The SDoH indicator scores 406, the actual indicator data, or other information may be included in the justification details 414 for the associated SDoH factor. According to another aspect, the justification engine 220 may determine a human-readable justification 412 comprised of one or more justifiers 416 associated with the one or more of the justification details 414 that describe each or a plurality of SDoH indicators factored into the SDoH factor propensity score 408. The justification 412 and justification details 414 may be stored in the SDoH database 218 and/or passed to the report generator 212.

At OPERATION 718, the method 700 may use the engagement strategy engine 208 to determine one or more engagement strategies 420 corresponding to SDoH factors that are identified as factors for which the one or more individuals 302 may have a propensity. For example, engagement strategies 420 and rule criteria for the engagement strategies may be stored in the engagement strategies rules database 210. In determining an engagement strategy 420 corresponding to an SDoH factor, the engagement strategy engine 208 may evaluate the justification 412 and justification details 414 associated with an SDoH factor propensity score 408 for determining if rule criteria for an engagement strategy 420 are met. If the criteria for an engagement strategy 420 are met, the engagement strategy engine 208 may determine that the engagement strategy is relevant to the individual 302 for addressing the associated SDoH factor. As described above, the engagement strategy 420 may be an effective reach-out or engagement approach that has been determined to be an effective means of addressing an SDoH factor need and positively impacting an individual's health outcome(s). The selected engagement strategies 420 may be stored in the SDoH database 218 and/or passed to the report generator 212.

At OPERATION 720, the method 700 may use the report generator 212 to access the outputs of the SDoH scoring engine 206, the justification engine 220, and the engagement strategy engine 208 for generating an SDoH propensity solution 402 for the one or more individuals 302. According to an aspect, the SDoH propensity solution 402 may include an overall propensity score 410 and propensity level 418, the justification 412 and justification details 414 for the overall propensity score, and one or more engagement strategies 420 that identifies, informs, and provides a solution for addressing the one or more individuals' SDoH. The SDoH propensity solution 402 is configured for transmission to a client device 102 and for display on the client device; the form of the SDoH propensity solution 402 and the delivery options may vary based on different software systems and/or client types. For example, if the SDoH request 304 for SDoH information for the one or more individuals is received in a batch 500 format, the report generator 212 may include the SDoH propensity solution 402 results in the batch report. As described above, the SDoH propensity solution 402 may include, for display, the determined overall propensity scores 410 and levels 418 in a way (e.g., use of color coding 502) that enables a client user to easily identify and act on a person's SDoH. Additionally, including the justification 412, justification details 414, and the engagement strategies 420 in the SDoH propensity solution 402 results can help make understanding the social determinants that impact an individual 302 more actionable.

At OPTIONAL OPERATION 722, the method 700 may use the UI engine 214 to generate a GUI 602 that displays the SDoH propensity solution 402 results. For example, if the SDoH request 304 for SDoH information for the one or more individuals is received via an API call, the report generator 212 may use the UI engine 214 to generate the GUI that can be integrated in a client application, platform, or software system for display of the SDoH propensity solution results to a client user. In some examples, the GUI 602 may be used to display aggregate profiles of SDoH across populations based on various selectable segments 618, which may help client users to understand the best areas or populations for population health investment initiatives.

At OPERATION 724, the SDoH propensity solution 402 may be transmitted to the client (e.g., in a batch 500 report or in a GUI 602) for display on the client device 102, and the method 700 may conclude at OPERATION 798.

FIG. 8 is a block diagram illustrating physical components of an example computing device with which aspects may be practiced. The computing device 800 may include at least one processing unit 802 and a system memory 804. The system memory 804 may comprise, but is not limited to, volatile (e.g. random access memory (RAM)), non-volatile (e.g. read-only memory (ROM)), flash memory, or any combination thereof. System memory 804 may include operating system 806, one or more program instructions 808, and may include sufficient computer-executable instructions for the SDoH system 104, which when executed, perform functionalities as described herein. Operating system 806, for example, may be suitable for controlling the operation of computing device 800. Furthermore, aspects may be practiced in conjunction with a graphics library, other operating systems, or any other application program and is not limited to any particular application or system. This basic configuration is illustrated by those components within a dashed line 810. Computing device 800 may also include one or more input device(s) 812 (keyboard, mouse, pen, touch input device, etc.) and one or more output device(s) 814 (e.g., display, speakers, a printer, etc.).

The computing device 800 may also include additional data storage devices (removable or non-removable) such as, for example, magnetic disks, optical disks, or tape. Such additional storage is illustrated by a removable storage 816 and a non-removable storage 818. Computing device 800 may also contain a communication connection 820 that may allow computing device 800 to communicate with other computing devices 822, such as over a network in a distributed computing environment, for example, an intranet or the Internet. Communication connection 820 is one example of a communication medium, via which computer-readable transmission media (i.e., signals) may be propagated.

Programming modules may include routines, programs, components, data structures, and other types of structures that may perform particular tasks or that may implement particular abstract data types. Moreover, aspects may be practiced with other computer system configurations, including hand-held devices, multiprocessor systems, microprocessor-based or programmable user electronics, minicomputers, mainframe computers, and the like. Aspects may also be practiced in distributed computing environments where tasks are performed by remote processing devices that are linked through a communications network. In a distributed computing environment, programming modules may be located in both local and remote memory storage devices.

Furthermore, aspects may be practiced in an electrical circuit comprising discrete electronic elements, packaged or integrated electronic chips containing logic gates, a circuit using a microprocessor, or on a single chip containing electronic elements or microprocessors (e.g., a system-on-a-chip (SoC)). Aspects may also be practiced using other technologies capable of performing logical operations such as, for example, AND, OR, and NOT, including, but not limited to, mechanical, optical, fluidic, and quantum technologies. In addition, aspects may be practiced within a general purpose computer or in any other circuits or systems.

Aspects may be implemented as a computer process (method), a computing system, or as an article of manufacture, such as a computer program product or computer-readable storage medium. The computer program product may be a computer storage medium readable by a computer system and encoding a computer program of instructions for executing a computer process. Accordingly, hardware or software (including firmware, resident software, micro-code, etc.) may provide aspects discussed herein. Aspects may take the form of a computer program product on a computer-usable or computer-readable storage medium having computer-usable or computer-readable program code embodied in the medium for use by, or in connection with, an instruction execution system.

Although aspects have been described as being associated with data stored in memory and other storage mediums, data can also be stored on or read from other types of computer-readable media, such as secondary storage devices, like hard disks, floppy disks, or a CD-ROM, or other forms of RAM or ROM. The term computer-readable storage medium refers only to devices and articles of manufacture that store data or computer-executable instructions readable by a computing device. The term computer-readable storage media do not include computer-readable transmission media.

Aspects of the present invention may be used in various distributed computing environments where tasks are performed by remote processing devices that are linked through a communications network.

Aspects of the invention may be implemented via local and remote computing and data storage systems. Such memory storage and processing units may be implemented in a computing device. Any suitable combination of hardware, software, or firmware may be used to implement the memory storage and processing unit. For example, the memory storage and processing unit may be implemented with computing device 800 or any other computing devices 822, in combination with computing device 800, wherein functionality may be brought together over a network in a distributed computing environment, for example, an intranet or the Internet, to perform the functions as described herein. The systems, devices, and processors described herein are provided as examples; however, other systems, devices, and processors may comprise the aforementioned memory storage and processing unit, consistent with the described aspects.

The description and illustration of one or more aspects provided in this application are intended to provide a thorough and complete disclosure the full scope of the subject matter to those skilled in the art and are not intended to limit or restrict the scope of the invention as claimed in any way. The aspects, examples, and details provided in this application are considered sufficient to convey possession and enable those skilled in the art to practice the best mode of the claimed invention. Descriptions of structures, resources, operations, and acts considered well-known to those skilled in the art may be brief or omitted to avoid obscuring lesser known or unique aspects of the subject matter of this application. The claimed invention should not be construed as being limited to any embodiment, aspects, example, or detail provided in this application unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently. Having been provided with the description and illustration of the present application, one skilled in the art may envision variations, modifications, and alternate embodiments falling within the spirit of the broader aspects of the general inventive concept provided in this application that do not depart from the broader scope of the present disclosure.

We claim:

1. A system for generating a social determinants of health (SDoH) propensity solution, comprising:
at least one processing device; and
at least one computer readable data storage device storing instructions that, when executed by the at least one processing device, cause the system to:
receive a request for information associated with an SDoH factor corresponding to an individual from a requestor, wherein the request includes identifying information about the individual;
determine, from the identifying information, a unique identifier for the individual;
based on a type of SDoH factor, identify indicators specific to the type of SDoH factor;
query a consumer marketing database using the unique identifier for consumer marketing data related to the individual and associated with the indicators, the consumer marketing data comprising non-clinical socio-economic data about the individual;
obtain additional data associated with the indicators from one or more other databases based at least in part on the non-clinical socio-economic data about the individual;
evaluate the consumer marketing data and the additional data associated with the indicators for determining an indicator score for each indicator;
determine, based on the indicator scores, a propensity score for the SDoH factor, wherein the propensity score indicates a likelihood of the SDoH factor having a negative impact on the individual's health outcome;
determine a justification for the propensity score;
determine an engagement strategy to reduce the negative impact of the SDoH factor on the individual's health outcome based on the justification;
generate an SDoH solution output that includes the propensity score, the justification, and the engagement strategy; and
transmit the SDoH solution output to the requestor.

2. The system of claim 1, wherein the SDoH factor includes a type of SDoH factor selected from a group of types of SDoH factors comprising:
accessibility to healthcare;
food insecurity;
housing instability;
interpersonal violence;
social isolation; and
healthcare literacy.

3. The system of claim 2, wherein in receiving the request, the system is configured to receive a request for information associated with a plurality of types of SDoH factors corresponding to the individual.

4. The system of claim 3, wherein the system is further configured to:
determine an indicator score for each indicator specific to the plurality of types of SDoH factors;
determine, based on the indicator scores, a propensity score for each of the plurality of types of SDoH factors; and
determine, based on the propensity scores, an overall propensity score for the individual.

5. The system of claim 2, wherein:
the type of SDoH factor is accessibility to healthcare;
the indicators include indicators specific to accessibility to healthcare.

6. The system of claim 5, wherein in obtaining the additional data, the system is configured to use residential and employment location information included in the consumer marketing data to obtain:
business data associated with health-related businesses within a search area of the individual's residential and employment locations; and
public transportation database, public transportation data associated with transit locations and routes.

7. The system of claim 6, wherein the non-clinical socio-economic data about the individual comprise at least one of:
the individual's age;
information about the individual's household composition;
the individual's residential location;
the individual's employment location;
profitability-related data; and
information about the individual's vehicle ownership.

8. The system of claim 7, wherein the propensity score indicating the likelihood of the SDoH factor having a negative impact on the individual's health outcome is determined based on the indicator scores of the indicators specific to accessibility to healthcare, the indicators comprising at least one of:
presence of a vehicle in the individual's household;
distance between the individual's residence location and a health-related business;
the individual's ability to afford gas money;
the individual's ability to walk;
the individual's ability to walk to a transit location;
the individual's ability to afford transit fare; and
the individual's ability to walk to a health-related business.

9. The system of claim 1, wherein:
the identifying information about the individual comprise demographic data; and
the identifier manger is configured to:
extract the demographic data from the request;
query a demographic database with the demographic data;
determine whether the demographic data matches with a demographic data set stored in the demographic database associated with a unique universal person identifier; and
in response to determining that the demographic data matches a given demographic data set stored in the demographic database associated with the unique universal person identifier:
map the unique universal person identifier to the unique identifier for the individual that can be used to obtain consumer marketing data about the individual.

10. The system of claim 1, wherein the system is further configured to:
determine a propensity level corresponding to the propensity score; and
include the propensity level in the SDoH solution output; and
apply color coding to the propensity score and propensity level for enabling a requesting user to easily identify whether the individual's health outcome is likely impacted by the SDoH factor.

11. The system of claim 10, wherein:
the request is received as part of a batch file that includes a listing of a plurality of individuals' identifying information for whom the request is requesting SDoH information; and
in generating the SDoH solution output, the system is configured to insert each individual's propensity score, justification for the propensity score, and engagement strategy in the batch file.

12. The system of claim 1, wherein:
the request is received via an application programming interface that is exposed by the system; and
in generating the SDoH solution output, the system is configured to generate a user interface including the individual's propensity score, justification for the SDoH factor propensity score, and engagement strategy.

13. The system of claim 12, wherein the user interface includes one or more data visualizations of aggregated profiles of SDoH across a population.

14. The system of claim 1, wherein in determining the justification, the system is further configured to:
determine justification details that correspond to the scores of the indicators;
determine justifiers corresponding to the justification details, wherein the justifiers are human-readable descriptors of the justification details;
designate whether each justifier supports or counters the individual's propensity for the SDoH factor; and
include the justifiers in the justification.

15. A method for providing generating a social determinants of health (SDoH) propensity solution, comprising:
receiving a request for information associated with an SDoH factor corresponding to an individual from a requestor, wherein the request includes identifying information about the individual;
determining, from the identifying information, a unique identifier for the individual;
based on a type of SDoH factor, identifying indicators specific to the type of SDoH factor;
querying a consumer marketing database using the unique identifier for consumer marketing data related to the individual and associated with the indicators, the consumer marketing data comprising non-clinical socio-economic data about the individual;
obtaining additional data associated with the indicators from one or more other databases based at least in part on the non-clinical socio-economic data about the individual;
evaluating the consumer marketing data and the additional data associated with the indicators for determining an indicator score for each indicator;
determining, based on the indicator scores, a propensity score for the SDoH factor, wherein the propensity score indicates a likelihood of the SDoH factor having a negative impact on the individual's health outcome;
determining a propensity level corresponding to the propensity score;
determining a justification for the propensity score;
determining an engagement strategy to reduce the negative impact of the SDoH factor on the individual's health outcome based on the justification;
generating an SDoH solution output that includes the propensity score, the propensity level, the justification, and the engagement strategy; and
transmitting the SDOH solution output to the requestor.

16. The method of claim 15, wherein receiving the request for information associated with an SDoH factor comprises receiving a request for information associated with at least one type of SDoH factor selected from a group of types of SDoH factors comprising:
accessibility to healthcare;
food insecurity;
housing instability;
interpersonal violence;
social isolation; and
healthcare literacy.

17. The method of claim 16, wherein:
receiving the request for information associated with an SDoH factor comprises receiving a request for information associated with accessibility to healthcare;
obtaining the additional data comprises using residential and employment location information included in the consumer marketing data to obtain:
business data associated with health-related businesses within a search area of the individual's residential and employment locations; and
public transportation database, public transportation data associated with transit locations and routes.

18. The method of claim 15, wherein:
receiving the request for information associated with an SDoH factor comprises receiving a request for information associated with an SDoH factor corresponding to a plurality of individuals; and
generating the SDoH solution output comprises inserting each of the plurality of individuals' propensity score, propensity level, justification for the propensity score, and engagement strategy in the SDoH solution output.

19. A computer-readable storage device including computer readable instructions, which when executed by a processing unit are configured to:
receive a request for information associated with an SDoH factor corresponding to an individual from a requestor, wherein the request includes identifying information about the individual;
determine, from the identifying information, a unique identifier for the individual that can be used to obtain consumer marketing data about the individual;
based on a type of SDoH factor, identify indicators specific to the type of SDoH factor;
using the unique identifier, obtain consumer marketing data related to the individual and associated with the indicators from a consumer marketing database, the consumer marketing data comprising non-clinical socio-economic data about the individual;
obtain additional data associated with the indicators from one or more other databases based at least in part on the non-clinical socio-economic data about the individual;
evaluate the consumer marketing data and the additional data associated with the indicators for determining an indicator score for each indicator;
determine, based on the indicator scores, a propensity score for the SDoH factor, wherein the propensity score indicates a likelihood of the SDoH factor having a negative impact on the individual's health outcome;
determine a propensity level corresponding to the propensity score;
determine a justification for the propensity score;
determine an engagement strategy to reduce the negative impact of the SDoH factor on the individual's health outcome based on the justification;
generate an SDoH solution output that includes the propensity score, the propensity level, the justification, and the engagement strategy; and
transmit the SDOH solution output to the requestor.

20. The computer-readable storage device of claim 19, wherein:
the type of SDoH factor is accessibility to healthcare;
as part of obtaining the additional data, the instructions are configured to use residential and employment location information included in the consumer marketing data to obtain:
business data associated with health-related businesses within a search area of the individual's residential and employment locations; and
public transportation database, public transportation data associated with transit locations and routes; and
the propensity score indicating the likelihood of the SDoH factor having a negative impact on the individual's health outcome is determined based on the indicator scores of the indicators specific to accessibility to healthcare, the indicators comprising at least one of:
presence of a vehicle in the individual's household;
distance between the individual's residence location and a health-related business;
the individual's ability to afford gas money;
the individual's ability to walk;
the individual's ability to walk to a transit location;
the individual's ability to afford transit fare; and
the individual's ability to walk to a health-related business.
